SEQUENCE LISTING (40 sequences)

SEQ ID NO: 1 - protein, 293 aa, Zymomonas mobilis
MTVFKHIAIIGLGLIGSSAARATKAYCPDVTVSLYDKSEFVCDRARALNLGDNVTDDIQDAVREADLVLLCVPVRAMGIVAAAMAPALKKDVIICDTGSVKVSVIKTLQDNLPNHIIVPSHPLAGTENNGPDAGFAELFQDHPVILTPDAHTPAQAIAYIADYWEEIGGRINLMSAEHHDHVLALTSHLPHVIAYQLIGMVSGYEKKSRTPIMRYSAGSFRDATRVAASEPRLWQDIMLENAPALLPVLDHFIADLKKLRTAIASQDEDYLLEHFKESQKARLALKTDHDIHP

SEQ ID NO: 2 - DNA, 882 bp, Zymomonas mobilis
atgaccgtctttaagcatattgccattatcggattaggactgatcggttcctctgcggcacgggcaacaaaggcctattgtcctgatgtaacggtcagtctctatgacaaaagcgaatttgtctgcgacagagctagagcgctcaatctcggcgacaatgtcaccgatgatattcaagatgcggttcgtgaggctgatctggtgctattatgcgtgccagtcagggcaatgggtatcgtcgcggcagcgatggcaccggcgctgaaaaaagacgttattatctgcgatacaggttcggtaaaagtcagcgttataaaaacgctgcaagacaatttacccaatcacattattgttcctagccatcctttggctgggactgaaaataacggacccgacgccggttttgctgaattattccaagaccatcctgttattttgacccccgatgcccatacaccggcacaggctatcgcctatatcgccgattattgggaagaaattggtgggcgtatcaatctgatgtcggcggaacatcacgatcacgttttagcgcttaccagccatttgcctcatgtcattgcataccaacttatagggatggtatcgggttatgagaaaaaaagccggacacccatcatgcgttattcggcaggcagctttcgggatgcgacgcgggtagcggcttcggaacctcgtctctggcaagatattatgctggaaaatgcgcctgctcttttaccagtgctggatcattttatcgcagatctcaaaaaattgcggacagctattgcttcgcaagatgaggattatcttcttgagcatttcaaagaatcgcagaaagcgcgtttagccttaaaaacagaccacgatattcacccttaa

SEQ ID NO: 3 - DNA, 140 bp, artificial sequence
  synthetic primer
tgaagaagagtagtcctttatattgagtgtatcgccaacgcgccttcgggcgcgtttttttgaaaaggtgccggatgatgtgaatcatccggcactggattattactggcgattgtcattttaagacccactttcacatt

SEQ ID NO: 4 - DNA, 140 bp, artificial sequence
  synthetic primer
aatgacaatcgccagtaataatccagtgccggatgattcacatcatccggcaccttttcaaaaaaacgcgcccgaaggcgcgttggcgatacactcaatataaaggactactcttcttcactaagcacttgtctcctgtt

SEQ ID NO: 5 - DNA, 20 bp, artificial sequence
  synthetic primer
ttaagacccactttcacatt

SEQ ID NO: 6 - DNA, 20 bp, artificial sequence
  synthetic primer
ctaagcacttgtctcctgtt

SEQ ID NO: 7 - DNA, 66 bp, artificial sequence
  synthetic primer
aattcatcaggatctgaacgggcagctgacggctcgcgtggcttaacgtcttgagcgattgtgtag

SEQ ID NO: 8 - DNA, 64 bp, artificial sequence
  synthetic primer
aaaacattatccagaacgggagtgcgccttgagcgacacgaatatgaatatcctccttagttcc

SEQ ID NO: 9 - DNA, 40 bp, artificial sequence
  synthetic primer
ctaaggaggatattcatattcgtgtcgctcaaggcgcact

SEQ ID NO: 10 - DNA, 66 bp, artificial sequence
  synthetic primer
cgacttcatcaatttgatcgcgtaatgcggtcaattcagcaaccatggtctgtttcctgtgtgaaa

SEQ ID NO: 11 - DNA, 26 bp, artificial sequence
  synthetic primer
caaccgcgcagtgaaatgaaatacgg

SEQ ID NO: 12 - DNA, 26 bp, artificial sequence
  synthetic primer
gcgctccggaacataaataggcagtc

SEQ ID NO: 13 - DNA, 27 bp, artificial sequence
  synthetic primer
cacctgatgaccgtctttaagcatatt

SEQ ID NO: 14 - DNA, 20 bp, artificial sequence
  synthetic primer
ttaagggcgaatatcgtggt

SEQ ID NO: 15 - DNA, 26 bp, artificial sequence
  synthetic primer
cacctgatggttgctgaattgaccgc

SEQ ID NO: 16 - DNA, 20 bp, artificial sequence
  synthetic primer
ttactggcgattgtcattcg

SEQ ID NO: 17 - DNA, 1122 bp, E. coli
atggttgctgaattgaccgcattacgcgatcaaattgatgaagtcgataaagcgctgctgaatttattagcgaagcgtctggaactggttgctgaagtgggcgaggtgaaaagccgctttggactgcctatttatgttccggagcgcgaggcatctatgttggcctcgcgtcgtgcagaggcggaagctctgggtgtaccgccagatctgattgaggatgttttgcgtcgggtgatgcgtgaatcttactccagtgaaaacgacaaaggatttaaaacactttgtccgtcactgcgtccggtggttatcgtcggcggtggcggtcagatgggacgcctgttcgagaagatgctgaccctctcgggttatcaggtgcggattctggagcaacatgactgggatcgagcggctgatattgttgccgatgccggaatggtgattgttagtgtgccaatccacgttactgagcaagttattggcaaattaccgcctttaccgaaagattgtattctggtcgatctggcatcagtgaaaaatgggccattacaggccatgctggtggcgcatgatggtccggtgctggggctacacccgatgttcggtccggacagcggtagcctggcaaagcaagttgtggtctggtgtgatggacgtaaaccggaagcataccaatggtttctggagcaaattcaggtctggggcgctcggctgcatcgtattagcgccgtcgagcacgatcagaatatggcgtttattcaggcactgcgccactttgctacttttgcttacgggctgcacctggcagaagaaaatgttcagcttgagcaacttctggcgctctcttcgccgatttaccgccttgagctggcgatggtcgggcgactgtttgctcaggatccgcagctttatgccgacatcattatgtcgtcagagcgtaatctggcgttaatcaaacgttactataagcgtttcggcgaggcgattgagttgctggagcagggcgataagcaggcgtttattgacagtttccgcaaggtggagcactggttcggcgattacgcacagcgttttcagagtgaaagccgcgtgttattgcgtcaggcgaatgacaatcgccagtaa

SEQ ID NO: 18 - protein, 373 aa, E. coli
MVAELTALRDQIDEVDKALLNLLAKRLELVAEVGEVKSRFGLPIYVPEREASMLASRRAEAEALGVPPDLIEDVLRRVMRESYSSENDKGFKTLCPSLRPVVIVGGGGQMGRLFEKMLTLSGYQVRILEQHDWDRAADIVADAGMVIVSVPIHVTEQVIGKLPPLPKDCILVDLASVKNGPLQAMLVAHDGPVLGLHPMFGPDSGSLAKQVVVWCDGRKPEAYQWFLEQIQVWGARLHRISAVEHDQNMAFIQALRHFATFAYGLHLAEENVQLEQLLALSSPIYRLELAMVGRLFAQDPQLYADIIMSSERNLALIKRYYKRFGEAIELLEQGDKQAFIDSFRKVEHWFGDYAQRFQSESRVLLRQANDNRQ

SEQ ID NO: 19 - DNA, 29 bp, artificial sequence
  synthetic primer
gagcgcgaggcatctattttggcctcgcg

SEQ ID NO: 20 - DNA, 29 bp, artificial sequence
  synthetic primer
cgcgaggccaaaatagatgcctcgcgctc

SEQ ID NO: 21 - DNA, 29 bp, artificial sequence
  synthetic primer
ccctctcgggttatcgggtgcggattctg

SEQ ID NO: 22 - DNA, 29 bp, artificial sequence
  synthetic primer
cagaatccgcacccgataacccgagaggg

SEQ ID NO: 23 - DNA, 29 bp, artificial sequence
  synthetic primer
gctacttttgctcacgggctgcacctggc

SEQ ID NO: 24 - DNA, 29 bp, artificial sequence
  synthetic primer
gccaggtgcagcccgtgagcaaaagtagc

SEQ ID NO: 25 - DNA, 35 bp, artificial sequence
  synthetic primer
ctggttcggcgattacgtacagcgttttcagagtg

SEQ ID NO: 26 - DNA, 35 bp, artificial sequence
  synthetic primer
cactctgaaaacgctgtacgtaatcgccgaaccag

SEQ ID NO: 27 - DNA, 29 bp, artificial sequence
  synthetic primer
ggagcgcgaggcatctatgttggcctcgc

SEQ ID NO: 28 - DNA, 29 bp, artificial sequence
  synthetic primer
gcgaggccaacatagatgcctcgcgctcc

SEQ ID NO: 29 - DNA, 17 bp, artificial sequence
  synthetic primer
cactttgtccgtcactg

SEQ ID NO: 30 - DNA, 17 bp, artificial sequence
  synthetic primer
cagtgacggacaaagtg

SEQ ID NO: 31 - DNA, 17 bp, artificial sequence
  synthetic primer
ggcgtttattcaggcac

SEQ ID NO: 32 - DNA, 17 bp, artificial sequence
  synthetic primer
gtgcctgaataaacgcc

SEQ ID NO: 33 - DNA, 17 bp, artificial sequence
  synthetic primer
gtaaaacgacggccagt

SEQ ID NO: 34 - DNA, 16 bp, artificial sequence
  synthetic primer
aacagctatgaccatg

SEQ ID NO: 35 - DNA, 24 bp, artificial sequence
  synthetic primer
cacctgatgggcgatattatgttt

SEQ ID NO: 36 - DNA, 18 bp, artificial sequence
  synthetic primer
ttattttttctgatccag

SEQ ID NO: 37 - DNA, 25 bp, artificial sequence
  synthetic primer
cacctgatgaacagcgcgccgatgt

SEQ ID NO: 38 - DNA, 18 bp, artificial sequence
  synthetic primer
ttattccgcgcctttgcc

SEQ ID NO: 39 - DNA, 24 bp, artificial sequence
  synthetic primer
cacctgatgaccaccgcgccgagc

SEQ ID NO: 40 - DNA, 18 bp, artificial sequence
  synthetic primer
ttacgcctgtttcgcttc

(19) United States
(12) Patent Application Publication
(10) Pub. No.: US 2008/0102499 A1
(43) Pub. Date: May 1, 2008

(54) Method of enhancing L-tyrosine production in recombinant bacteria

(76) Inventors: Lori Jean Templeton, Woodbury, NJ (US); Tina K. Van Dyk, Wilmington, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(21) Appl. No.: 11/588,994

(22) Filed: Oct. 27, 2006

(51) Int. Cl. C12P 13/22; C12N 1/21

(52) U.S. Cl. 435/108; 435/252.33

(57) ABSTRACT

Tyrosine production in a tyrosine over-producing enteric bacterial strain was enhanced by expression of a tyrosine insensitive prephenate dehydrogenase. The prephenate dehydrogenase expressed was the cyclohexadienyl dehydrogenase encoded by the Zymomonas mobilis tyrc gene.

### FIELD OF THE INVENTION

The invention relates to the field of molecular biology and microbiology. More specifically, the invention relates to methods of engineering bacterial hosts for enhanced L-tyrosine production by expressing a tyrosine insensitive prephenate dehydrogenase.

### BACKGROUND OF THE INVENTION

Production of chemicals from microorganisms has been an important application of biotechnology. Tyrosine is an attractive chemical for production in microorganisms due to its nutritional and pharmaceutical uses, such as being a dietary supplement and a reagent for production of the anti-Parkinson's drug, L-DOPA. In addition, tyrosine has potential as a reagent for the production of other chemicals with valuable industrial applications. Compounds that may potentially be made from tyrosine include (S)-4-(2-chloro-3-(4-n-dodecyloxy)-phenylpropionato)-4′4(2-methyl)butyloxy-biphenylcarboxylate (CDPMBB; Kumar and Pisipati (Z. Naturforsch. 57a:803-806 (2002)), p-hydroxycinnamic (pHCA; U.S. Pat. No. 6,368,837, US 20050148054A1), p-hydroxystyrene (pHS; also know as p-vinylphenol; US 2004001860), and acetylated derivatives thereof, such as p-acetoxystyrene (also known as ASM). CDPMBB is a ferroelectric material for use in ferroelectric liquid crystals (FLC). PHCA is a useful monomer for production of Liquid Crystal Polymers (LCP). LCPs may be used in electronic connectors and telecommunication and aerospace applications. LCP resistance to sterilizing radiation has also enabled these materials to be used in medical devices as well as chemical, and food packaging applications. Hydroxystyrenes have application as monomers for the production of resins, elastomers, adhesives, coatings, automotive finishes, inks and photoresists, as well as in electronic materials. They may also be used as additives in elastomer and resin formulations.

Tyrosine is made naturally in microorganisms, but is generally present at low levels that are sufficient for cellular growth. The tyrosine biosynthetic pathway branches from the phenylalanine biosynthetic pathway with the chorismate mutase/prephenate dehydrogenase enzyme, encoded by tyrA in E. coli, acting on the chorismate substrate. In the phenylalanine pathway chorismate is the substrate of chorismate mutase/prephenate dehydratase, which is encoded by the pheA gene in E. coli.

Microorganisms with increased levels of tyrosine production have been obtained through traditional genetic methods as well as through genetic engineering. Expression of either pheA, or the genes encoding chorismate mutase/prephenate dehydratase in other organisms, has been reduced or eliminated, thereby reducing or eliminating competition for the chorismate substrate by chorismate mutase/prephenate dehydratase, resulting in increased tyrosine production [Maiti et al. (1995) Microbial production of L-tyrosine: a review. Hindustan Antibiot. Bull. 37:51-65].

Separately, either tyrA expression or the genes encoding chorismate mutase/prephenate dehydrogenase in other organisms, has been increased thereby increasing the cellular capacity to direct chorismate toward tyrosine production, with increased chorismate mutase/prephenate dehydrogenase enzyme activity. EP 0332234 discloses a process for producing tyrosine in a Corynebacterium or Brevibacterium host by transforming with a plasmid carrying genes encoding 3-deoxy-2-keto-D-arabino-heptulosonate-7phosphate (DAHP) synthase (first enzyme of the aromatic amino acid biosynthetic pathway), chorismate mutase, and prephenate dehydrogenase. EP 0263515 discloses a process for producing tyrosine in a Corynebacterium or Brevibacterium host that produces tryptophan. The tryptophan producing Corynebacterium or Brevibacterium host is transformed with a plasmid carrying genes encoding DAHP synthase and chorismate mutase.

Commonly owned US 20040248267 discloses engineering of a tyrosine excreting E. coli strain by first introducing a mutant pheA gene. Then in a second separate step, a trc promoter driven tyrA gene was introduced. Rare transductants having both introductions were identified as tyrosine excreting strains. Commonly owned and co-pending U.S. application Ser. No. 11/448,331 discloses a rapid method for creating a tyrosine over-producing strain by manipulating these two genes in one step.

In addition, commonly owned US 20050148054 A1 discloses increasing tyrosine production by expressing phenylalanine hydroxylase in a recombinant organism to convert phenylalanine to tyrosine.

In spite of the efforts to redirect flow in the aromatic amino acid biosynthesis pathway from phenylalanine to tyrosine, some phenylalanine is still synthesized in engineered tyrosine over-producing strains, which lack pheA expression (Pittard, A. J. 1996. Biosynthesis of aromatic amino acids. In F. C. Neidhardt (ed.), Escherichia coli and Salmonella: Cellular and Molecular Biology. ASM Press, Washington, D.C.). There remains a need to engineer strains for reduced phenylalanine synthesis to provide increased tyrosine synthesis. Applicants have solved the stated problem by engineering a recombinant enteric bacteria that produces less phenylalanine and increased L-tyrosine.

### SUMMARY OF THE INVENTION

The invention relates to a recombinant host cell engineered to provide expression of a tyrosine insensitive prephenate dehydrogenase, and a method of producing tyrosine using the engineered cell. The engineered cell shows enhanced tyrosine synthesis with reduced phenylalanine synthesis. Accordingly the invention provides an enhanced enteric tyrosine over-producing recombinant host cell comprising a genetic construct encoding a heterologus tyrosine insensitive prephenate dehydrogenase. The host cell additionally comprises other modulations of the aromatic amino acid pathway and other phenotypic traits that enhance the utility of the strain for the production of tyrosine.

In another embodiment the invention provides a method for producing L-tyrosine comprising:
a) providing an enhanced tyrosine over-producing enteric bacterial strain comprising a tyrosine insensitive prephenate dehydrogenase enzyme; and
b) growing said enhanced tyrosine over-producing strain under conditions where L-tyrosine is produced.

### BRIEF DESCRIPTION OF THE DRAWINGS AND SEQUENCE DESCRIPTIONS

The invention can be more fully understood from the following detailed description, the figures, and the accompanying sequence descriptions that form a part of this application.

FIG. 1 is an illustration of the aromatic amino acid biosynthetic pathway.

The following sequences conform with 37 C.F.R. 1.821-1.825 (“Requirements for Patent Applications Containing Nucleotide Sequences and/or Amino Acid Sequence Disclosures—the Sequence Rules”) and consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

A Sequence Listing is provided herewith on Compact Disk. The contents of the Compact Disk containing the Sequence Listing are hereby incorporated by reference in compliance with 37 CFR 1.52(e). The Compact Disks are submitted in triplicate and are identical to one another. The disks are labeled “Copy 1—Sequence Listing”, “Copy 2—Sequence Listing”, and CRF. The disks contain the following file: CL3286 Seqs.ST25 having the following size: 16,000 bytes and which was created Oct. 26, 2006.

SEQ ID NO:1 is the amino acid sequence of Z. mobilis TyrC protein.

SEQ ID NO:2 is the nucleotide sequence of the Z. mobilis tyrC coding region used for expression.

SEQ ID NO:3 is the nucleotide sequence of primer ABTR.

SEQ ID NO:4 is the nucleotide sequence of primer BATA.

SEQ ID NO:5 is the nucleotide sequence of primer TR.

SEQ ID NO:6 is the nucleotide sequence of primer TA.

SEQ ID NO:7 is the nucleotide sequence of primer T-kan(tyrA).

SEQ ID NO:8 is the nucleotide sequence of primer B-kan(trc).

SEQ ID NO:9 is the nucleotide sequence of primer T-trc(kan).

SEQ ID NO:10 is the nucleotide sequence of primer B-trc(tyrA).

SEQ ID NO:11 is the nucleotide sequence of primer T-ty(test).

SEQ ID NO:12 is the nucleotide sequence of primer B-ty(test).

SEQ ID NOs:13, 14 are the nucleotide sequences of primers for PCR of the Z. mobilis tyrC coding region.

SEQ ID NOs:15, 16 are the nucleotide sequence of primers for PCR of the for E. coli K12 tyrA coding region.

SEQ ID NO:17 is the nucleotide sequence of the E. coli K12 tyrA coding region.

SEQ ID NO:18 is the amino acid sequence of the E. coli K12 TyrA protein.

SEQ ID NOs:19, 20 are the nucleotide sequences of mutagenesis primers used to create M531.

SEQ ID NOs:21, 22 are the nucleotide sequences of mutagenesis primers used to create Q124R.

SEQ ID NOs:23, 24 are the nucleotide sequences of mutagenesis primers used to create Y263H.

SEQ ID NOs:25, 26 are the nucleotide sequences of mutagenesis primers used to create A354V.

SEQ ID NOs:27, 28 are the nucleotide sequences of mutagenesis primers used to create T51A.

SEQ ID NOs:29-34 are the nucleotide sequences of primers used to sequence tyrA mutants.

SEQ ID NOs:35, 36 are the nucleotide sequences of primers for PCR of the coding region for Agrobacterium tumefaciens TyrC related protein.

SEQ ID NOs:37, 38 are the nucleotide sequences of primers for PCR of the coding region for Rhodopseudomonas palustris TyrC related protein.

SEQ ID NOs:39, 40 are the nucleotide sequences of primers for PCR of the coding region for Rhodospirillum rubrum TyrC related protein.

### DETAILED DESCRIPTION

The present invention provides a strain of enteric bacteria that is engineered to express a tyrosine insensitive prephenate dehydrogenase enzyme. In particular, a gene directing expression of a tyrosine insensitive prephenate dehydrogenase is introduced into a tyrosine over-producing enteric bacterial strain. The added enzyme activity reduces phenylalanine production and further increases tyrosine production.

Tyrosine has nutritional and pharmaceutical uses, such as being a dietary supplement and a reagent for production of the anti-Parkinson's drug, L-DOPA. In addition, tyrosine has potential as a reagent for the production of other chemicals with valuable industrial applications.

The following abbreviations and definitions will be used for the interpretation of the specification and the claims.

“Polymerase chain reaction” is abbreviated PCR.

“Ampicillin” is abbreviated amp.

“Kanamycin is abbreviated kan.

The term “invention” or “present invention” as used herein shall not be limited to any particular embodiment of the invention but shall refer to all the varied embodiments described by the specification ad the claims.

“Gene” refers to a nucleic acid fragment that expresses a specific protein, including regulatory sequences preceding (5′ non-coding sequences) and following (3′ non-coding sequences) the coding sequence. “Native gene” or “wild type gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. The term “open reading frame” refers to that portion of a gene or genetic construct that encodes a polypeptide but may be devoid of any regulatory elements.

The term “deletion” or “disruption” when used in reference to a gene, genetic construct or the like with refer to the partial or complete inactivation of nucleic acid sequence as it normally functions. A deletion in a sequence means the removal of all or part of the sequence which may results in the complete or partial inactivation of the sequence. A disruption or insertion in the sequence will refer the addition of an element within the sequence that will again decrease or eliminate the ability of the sequence to function normally. Deletions, or disruptions will render the gene or coding sequence “non-functional” within the meaning the present invention.

“Coding sequence” or “coding region” refers to a DNA sequence that codes for a specific amino acid sequence.

“Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters which cause a gene to be expressed in most cell types at most times are commonly referred to as “constitutive promoters”. It is further recognized that since in most cases the exact boundaries of regulatory sequences have not been completely defined, DNA-fragments of different lengths may have identical promoter activity.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding operably linked to regulatory sequences in sense or antisense orientation.

The “3′ non-coding sequences” or “termination control region” or “terminator” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

The term “genetic construct” refers to a nucleic acid fragment that encodes for expression of one or more specific proteins. In the gene construct the gene may be native, chimeric, or foreign in nature. Typically a genetic construct will comprise a “coding sequence”. A “coding sequence” refers to a DNA sequence that codes for a specific amino acid sequence.

As used herein, the terms “isolated nucleic acid molecule” and “isolated nucleic acid fragment” are used interchangeably and mean a polymer of RNA or DNA that is single- or double-stranded, optionally containing synthetic, non-natural or altered nucleotide bases. An isolated nucleic acid molecule in the form of a polymer of DNA may be comprised of one or more segments of cDNA, genomic DNA or synthetic DNA.

The term “expression”, as used herein, refers to the transcription and stable accumulation of sense (mRNA) or antisense RNA derived from the nucleic acid fragment of the invention. Expression may also refer to translation of mRNA into a polypeptide.

The term “overexpression” refers to the production of a gene product in transgenic organisms that exceeds levels of production in normal or non-transformed organisms.

The term “messenger RNA (mRNA)” as used herein, refers to the RNA that is without introns and that can be translated into protein by the cell.

“Transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

The terms “plasmid”, and “vector” refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

The term “host cell” refers to a cell that contains a plasmid or a vector and supports the replication or expression of the plasmid or the vector. Alternatively, foreign DNA may be may be integrated into the genome of a host cell.

The term “tyrosine insensitive” refers to an enzyme activity that is reduced by less than 50% in the presence of 2 mM of tyrosine.

“pheA” refers to a gene found in an enteric bacteria encoding chorismate mutase/prephenate dehydratase and PheA refers to the corresponding encoded protein.

“tyrA” refers a gene found in enteric bacteria encoding chorismate mutase/prephenate dehydrogenase, and TyrA refers to the corresponding encoded protein.

“tyrR” refers a gene found in enteric bacteria that regulates the expression of various elements of the aromatic amino acid biosynthetic pathway including the gene products of the aroF, tyrA, aroG, aroL, and tyrB genes.

“PEP” is the abbreviation for Phosphoenolpyruvate

“DAHP” is the abbreviation for 3-deoxy-D-arabino-heptulosonate 7-phosphate

“DHQ” is the abbreviation for Dehydroquinate

“DHS” is the abbreviation for Dehydroshikimate

“SHK” is the abbreviation for Shikimate

“S-3P” is the abbreviation for shikimate-3-phosphate.

“ESPS is the abbreviation for Enolether-5-enolpyruvylshikimate-3-phosphate.

“CHA” is the abbreviation for chorismate.

“PPA” is the abbreviation for prephenate

“HPP” is the abbreviation for 4-OH-phenylpyruvate

“Tyr” is the abbreviation for tyrosine

“Phe” is the abbreviation for phenylalanine.

The term “cyclohexadienyl dehydrogenase” refers to enzymes that are able to dehydrogenate both arogenate and prephenate. Thus a cyclohexadienyl dehydrogenase is an arogenate dehydrogenase and a prephenate dehydrogenase.

The term “prephenate dehydrogenase” refers to enzymes that are able to dehydrogenate prephenate. A cyclohexadienyl dehydrogenase is also a prephenate dehydrogenase, since prephenate is one of its substrates.

The term “aroG397” refers to a specific mutation in the aroG gene that results in the production of a DAHP synthase enzyme that is resistant to feed back inhibition by phenylalanine. The aroG397 mutation is common and well known in the art and is documented in U.S. Pat. No. 4,681,852, incorporated herein by reference.

As used herein the term “tyrR366 mutation” has the effect of inactivating, down regulating, or making non-functional the tyrR gene. Within the context of the present methods for the production of tyrosine, down regulation of tyrR results in the upregulation of a number of the enzymes of the aromatic biosynthetic pathway for which TyrR represses expression. The tyrR366 mutation is well known in the art and is well documented in [Camakaris and Pittard (1973) J. Bacteriol. 115: 1135-1144].

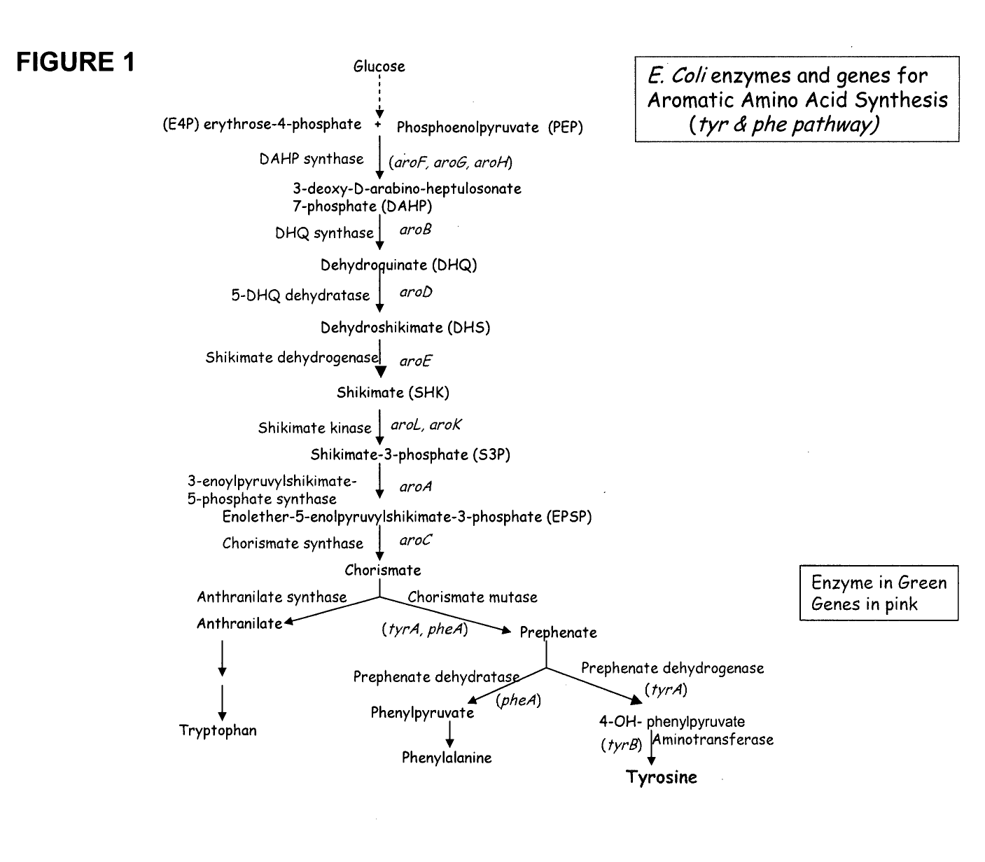

The term “aromatic amino acid biosynthetic pathway” refers to a ubiquitous enzymatic pathway found in many microorganisms responsible for phenylalanine and tyrosine production. As used herein the aromatic amino acid biosynthetic pathway is illustrated in FIG. 1 and, in part, comprises the enzymes encoded by the genes aroF, aroG, aroH, aroB, aroD, aroE, aroL, aroK, aroA, aroC, tyrA, pheA and tyrB

The term “phenylalanine over-producing strain” refers to a microbial strain that produces endogenous levels of phenylalanine that are significantly higher than those seen in the wildtype of that strain. One specific example of an E. coli phenylalanine over-producer is the E. coli strain NST74 (U.S. Pat. No. 4,681,852). Others may include Corynebacterium glutamicum [Ikeda, M. and Katsumata, R. Metabolic engineering to produce tyrosine or phenylalanine in a tryptophan-producing Corynebacterium glutamicum strain, Appl. Environ. Microbiol. (1992), 58(3), pp. 781-785]. When produced at high levels, phenylalanine is typically excreted into the medium, and thus a phenylalanine over-producing strain is generally also a “phenylalanine excreting strain”.

The term “tyrosine over-producing strain” refers to a microbial strain that produces endogenous levels of tyrosine that are significantly higher than those seen in the wildtype of that strain. When produced at high levels, tyrosine is typically excreted into the medium, and thus a tyrosine over-producing strain is generally also a “tyrosine excreting strain”.

“Tyrosine” refers to L-tyrosine, “phenylalanine” refers to L-phenylalanine, and “tryptophan” refers to L-tryptophan. These are the L-isomers of the named compounds.

The term “marker” means a gene that confers a phenotypic trait that is easily detectable through screening or selection. A selectable marker is one wherein cells having the marker gene can be distinguished based on growth. For example, an antibiotic resistance marker serves as a useful selectable marker, since it enables detection of cells which are resistant to the antibiotic, when cells are grown on media containing that particular antibiotic. A marker used in screening is, for example, one whose conferred trait can be visualized. Genes involved in carotenoid production or that encode proteins (i.e. beta-galactosidase, beta-glucuronidase) that convert a colorless compound into a colored compound are examples of this type of marker. A screening marker gene may also be referred to as a reporter gene.

The term “making use of the marker” means identifying cells based on the phenotypic trait provided by the marker. The marker may provide a trait for identifying cells by methods including selection and screening.

The term “negative selection marker” means a DNA sequence which confers a property that is detrimental under particular conditions. The property may be detrimental to a plasmid or to a whole cell. For example, expression of a sacB gene in the presence of sucrose is lethal to the expressing cells. Another example is a temperature sensitive origin of replication, which is nonfunctional at nonpermissive temperature such that the plasmid cannot replicate.

A nucleic acid molecule is “hybridizable” to another nucleic acid molecule, such as a cDNA, genomic DNA, or RNA molecule, when a single-stranded form of the nucleic acid molecule can anneal to the other nucleic acid molecule under the appropriate conditions of temperature and solution ionic strength. Hybridization and washing conditions are well known and exemplified in Sambrook, J., Fritsch, E. F. and Maniatis, T. Molecular Cloning: A Laboratory Manual, 2^{nd} ed., Cold Spring Harbor Laboratory: Cold Spring Harbor, N.Y. (1989), particularly Chapter 11 and Table 11.1 therein (entirely incorporated herein by reference). The conditions of temperature and ionic strength determine the “stringency” of the hybridization. Stringency conditions can be adjusted to screen for moderately similar fragments (such as homologous sequences from distantly related organisms), to highly similar fragments (such as genes that duplicate functional enzymes from closely related organisms). Post-hybridization washes determine stringency conditions. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions uses higher temperatures in which the washes are identical to those above except for the temperature of the final two 30 min washes in 0.2×SSC, 0.5% SDS was increased to 60° C. Another preferred set of highly stringent conditions uses two final washes in 0.1×SSC, 0.1% SDS at 65° C. An additional set of stringent conditions include hybridization at 0.1×SSC, 0.1% SDS, 65° C. and washed with 2×SSC, 0.1% SDS followed by 0.1×SSC, 0.1% SDS, for example.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al., supra, 9.50-9.51). For hybridizations with shorter nucleic acids, i.e., oligonucleotides, the position of mismatches becomes more important, and the length of the oligonucleotide determines its specificity (see Sambrook et al., supra, 11.7-11.8). In one embodiment the length for a hybridizable nucleic acid is at least about 10 nucleotides. Preferably a minimum length for a hybridizable nucleic acid is at least about 15 nucleotides; more preferably at least about 20 nucleotides; and most preferably the length is at least about 30 nucleotides. Furthermore, the skilled artisan will recognize that the temperature and wash solution salt concentration may be adjusted as necessary according to factors such as length of the probe.

The term “percent identity”, as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in: 1.) Computational Molecular Biology (Lesk, A. M., Ed.) Oxford University: NY (1988); 2.) Biocomputing: Informatics and Genome Projects (Smith, D. W., Ed.) Academic: NY (1993); 3.) Computer Analysis of Sequence Data, Part I (Griffin, A. M., and Griffin, H. G., Eds.) Humania: NJ (1994); 4.) Sequence Analysis in Molecular Biology (von Heinje, G., Ed.) Academic (1987); and 5.) Sequence Analysis Primer (Gribskov, M. and Devereux, J., Eds.) Stockton: NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignment of the sequences is performed using the Clustal method of alignment (Higgins and Sharp. CABIOS. 5:151-153 (1989)) with default parameters (GAP PENALTY=10, GAP LENGTH PENALTY=10). Default parameters for pairwise alignments using the Clustal method are: KTUPLE 1, GAP PENALTY=3, WINDOW=5 and DIAGONALS SAVED=5.

The term “codon degeneracy” refers to the degeneracy in the genetic code permitting variation of the nucleotide sequence without effecting the amino acid sequence of an encoded polypeptide.

The skilled artisan is well aware of the “codon-bias” exhibited by a specific host cell in usage of nucleotide codons to specify a given amino acid. Therefore, when synthesizing a gene for improved expression in a host cell, it is desirable to design the gene such that its frequency of codon usage approaches the frequency of preferred codon usage of the host cell.

The term “amino acid” refers to the basic chemical structural unit of a protein or polypeptide. The following abbreviations will be used herein to identify specific amino acids:

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Asparagine or aspartic acid | Asx | B |
| Cysteine | Cys | C |
| Glutamine | Gln | Q |
| Glutamic acid | Glu | E |
| Glutamine or glutamic acid | Glx | Z |
| Glycine | Gly | G |
| Histidine | His | H |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The term “chemically equivalent amino acid” refers to an amino acid that may be substituted for another in a given protein without altering the chemical or functional nature of that protein. For example, it is well known in the art that alterations in a gene which result in the production of a chemically equivalent amino acid at a given site, but do not effect the functional properties of the encoded protein are common. For the purposes of the present invention substitutions are defined as exchanges within one of the following five groups:
1. Small aliphatic, nonpolar or slightly polar residues: Ala, Ser, Thr (Pro, Gly);
2. Polar, negatively charged residues and their amides: Asp, Asn, Glu, Gin;
3. Polar, positively charged residues: His, Arg, Lys;
4. Large aliphatic, nonpolar residues: Met, Leu, Ile, Val (Cys); and
5. Large aromatic residues: Phe, Tyr, Trp.

Thus, alanine, a hydrophobic amino acid, may be substituted by another less hydrophobic residue (such as glycine) or a more hydrophobic residue (such as valine, leucine, or isoleucine). Similarly, changes which result in substitution of one negatively charged residue for another (such as aspartic acid for glutamic acid) or one positively charged residue for another (such as lysine for arginine) can also be expected to produce a functionally equivalent product. Each of the proposed modifications is well within the routine skill in the art, as is determination of retention of biological activity of the encoded products, and may be present in “substantially similar” proteins. Additionally, in many cases, alterations of the N-terminal and C-terminal portions of the protein molecule would also not be expected to alter the activity of the protein, and may occur in substantially similar proteins.

The term “sequence analysis software” refers to any computer algorithm or software program that is useful for the analysis of nucleotide or amino acid sequences. “Sequence analysis software” may be commercially available or independently developed. Typical sequence analysis software will include, but is not limited to: the GCG suite of programs (Wisconsin Package, Genetics Computer Group (GCG), Madison, Wis.), BLASTP, BLASTN, BLASTX (Altschul et al., J. Mol. Biol. 215:403-410 (1990)), DNASTAR (DNASTAR, Inc., Madison, Wis.), and the FASTA program incorporating the Smith-Waterman algorithm (W. R. Pearson, Comput. Methods Genome Res., [Proc. Int. Symp.] (1994), Meeting Date 1992, 111-20. Editor(s): Suhai, Sandor. Publisher: Plenum, New York, N.Y.). Within the context of this application it will be understood that where sequence analysis software is used for analysis, the results of the analysis will be based on the “default values” of the program referenced, unless otherwise specified. As used herein “default values” will mean any set of values or parameters that originally load with the software when first initialized. More preferred amino acid fragments are those that are at least about 90% identical to the sequences herein using a BLASTP analysis, where about 95% is preferred. Similarly, preferred nucleic acid sequences corresponding to the sequences herein are those encoding active proteins and which are at least 90% identical to the nucleic acid sequences reported herein. More preferred nucleic acid fragments are at least 95% identical to the sequences herein.

Standard recombinant DNA and molecular cloning techniques used here are well known in the art and are described by Sambrook, J., Fritsch, E. F. and Maniatis, T., Molecular Cloning: A Laboratory Manual, 2^{nd} ed., Cold Spring Harbor Laboratory: Cold Spring Harbor, N.Y. (1989) (hereinafter “Maniatis”); and by Silhavy, T. J., Bennan, M. L. and Enquist, L. W., Experiments with Gene Fusions, Cold Spring Harbor Laboratory: Cold Spring Harbor, N.Y. (1984); and by Ausubel, F. M. et al., Current Protocols in Molecular Biology, published by Greene Publishing Assoc. and Wiley-Interscience, Hoboken, N.J. (1987).

The present invention provides enhanced tyrosine producing enteric bacterial strains and a method of producing of L-tyrosine by fermentation using the enhanced strains. Applicants have found that expression of an enzyme with prephenate dehydrogenase activity, that is insensitive to tyrosine feedback inhibition, in a tyrosine over-producing strain reduces the amount of phenylalanine and increases the amount of tyrosine synthesized. An enzyme with prephenate dehydrogenase activity is able to catalyze the conversion of prephenate to 4-OH-phenylpyruvate, which can be converted by an aminotransferase to tyrosine (see FIG. 1). A prephenate dehydrogenase that is also a cyclohexadienyl dehydrogenase was used. A cyclohexadienyl dehydrogenase also has activity that converts arogenate to tyrosine. Upon prephenate accumulation, arogenate may be formed by transamination of prephenate.

### Tyrosine Over-Producing Host Strain

In the present invention, a tyrosine insensitive prephenate dehydrogenase is expressed in a tyrosine over-producing strain to further increase the amount of tyrosine produced. Tyrosine over-producing strains are known, and new strains may be engineered for tyrosine over-production by modifications of key elements of the aromatic amino acid pathway. The relevant elements of the aromatic amino acid pathway are illustrated in FIG. 1. Briefly, the pathway receives carbon ultimately from glucose and synthesis proceeds with the condensation of E4P and PEP to form DAHP, catalyzed by DAHP synthase, which is encoded by the aroFGH set of genes. The pathway proceeds though various intermediates catalyzed by the enzymes encoded to the genes aroB, aroD, aroE, aroL, aroK, aroA and aroC, as shown in FIG. 1, to the point where chorismate is produced. Chorismate is a substrate for both anthranilate synthase (leading to trytophan synthesis) and chorismate mutase leading to the synthesis of first prephenate which itself may be acted on by prephenate dehydratase (encoded by pheA) leading to phenylalanine synthesis, or prephenate deydrogenase (encoded by tyrA) leading first to the production of 4-OH-phenylpyruvate and then to tyrosine via catalysis by the tyrB encoded aminotransferase.

Given the elements of the pathway it will be apparent that maximizing tyrosine production involves control of the loss of carbon to competing products (phenylalanine, tryptophan) and optimizing carbon flow toward the tyrosine product. Thus, up-regulation of the gene product of tyrA and elimination of gene product of pheA are indicated. Additionally, because wildtype DAHP synthases are known to be inhibited by the end products of the pathway (phenylalanine, tryptophan, tyrosine), and because this is the first enzyme in the pathway controlling carbon flow, tyrosine over-producing strains may contain a mutant DAHP synthase with decreased regulation by end product. For example, E. coli has three isozymes of this enzyme encoded by aroG, aroF, and aroH. In wildtype E. coli, the aroG-encoded enzyme is inhibited by phenylalanine, the aroF-encoded enzyme is inhibited by tyrosine, and the aroH-encoded enzyme is inhibited by tryptophan. Thus, any of these isozymes may be altered to confer feedback resistance. The aroG397 mutation, disclosed in U.S. Pat. No. 4,681,852, (incorporated herein by reference) is particularly useful in creating a feedback resistant DAHP enzyme. TyrR is a regulatory protein that represses the expression of several genes, including aroF, tyrA, aroG, aroL, and tyrB, in the aromatic amino acid biosynthetic pathway [Pittard et al. (2005) Mol. Microbiol. 55:16-26]. Thus rendering TyrR nonfunctional, either through mutation in the protein or by blocking expression of the tyrR gene, disclosed in U.S. Pat. No. 4,681,852, may be desired in a tyrosine over-producing strain. The tyrR366 mutation [Camakaris and Pittard (1973) J. Bacteriol. 115: 1135-1144] is particularly useful for inactivating TyrR. Thus eliminating the repression effect of TyrR, as well as making DAHP synthase feedback resistant, creates more flow of intermediates through the aromatic amino acid biosynthetic pathway to chorismate, which is particularly useful in a tyrosine over-producing strain for use in the present invention.

Typically a tyrosine over-producing strain has the ability to produce chorismate via the aromatic amino acid biosynthetic pathway, has a non-functional pheA gene, and over-expresses tyrA. Disruption of pheA has the effect of blocking carbon to the production of phenylalanine (FIG. 1) and the over-expression of tyrA moves this additional carbon into the part of the pathway dedicated to tyrosine production (FIG. 1). The tyrosine over-producing strain may have a variety of other genetic and phenotypic traits, including but not limited to, a gene encoding a DAHP synthase resistant to feedback inhibition by phenylalanine, down regulation of the tyrR gene; and over-expression of aroF, aroG, aroH, aroB, aroD, aroE, aroK, aroL, aroA, aroC and/or tyrB genes. In addition, resistances to pathway products and analogs of pathway products can enhance tyrosine production. Compounds such as 3-fluorotyrosine, para-fluorophenylalanine, β-2-thienylalanine, tyrosine, and phenylalanine may each be used in screens for resistant cells. As used herein the term “resistance” as applied to the above mentioned compounds is used in a manner consistent with protocols for cell mutagenesis and screening for resistance to these compounds as described in U.S. Pat. No. 4,681,852, incorporated herein by reference. Cells resistant to aromatic amino acid biosynthetic pathway products and analogs of pathway products may have mutations that affect DAHP feedback resistance, TyrR regulation, or other pathway flow controlling factors. The specific mutations that cause the resistance properties need not be completely characterized in order for the cells containing the mutations to be useful in making tyrosine over-producing strains.

Strains that demonstrate robust production of phenylalanine, indicating a complete and enhanced aromatic amino acid pathway, are particularly useful in preparing tyrosine over-producing strains. Specific examples of E. coli phenylalanine over-producers are the E. coli K12 strains NST37 (ATCC #31882) and NST74 (ATCC #31884), both described in U.S. Pat. No. 4,681,852, incorporated herein by reference. An example of a non-K12 E. coli strain with low levels of phenylalanine excretion that may be converted to a tyrosine over-producer is ATCC#13281 (U.S. Pat. No. 2,973,304), incorporated herein by reference. Strains may be converted to tyrosine over-producers in a one-step process by integrating a chromosomal segment that includes a disrupted pheA gene and a tyrA over-expression gene as described in co-owned and co-pending U.S. patent application Ser. No. 11/448,331, incorporated herein by reference. Particularly useful tyrosine over-producing strains for the present invention include E. coli TY1, available from OmniGene Bioproducts, Inc. (Cambridge, Mass.); E. coli DPD4009, described in US 20050/260724, which is herein incorporated by reference, E. coli DPD4515, described in US 20050260724 A1, which is herein incorporated by reference; and E. coli DPD4119 and E. coli DPD4145, described in commonly owned and co-pending U.S. patent application Ser. No. 11/448,331.

Strains particularly useful in the present invention are those belonging to the class of enteric bacteria. Enteric bacteria are members of the family Enterobacteriaceae, and include such members as Escherichia, Salmonella, and Shigella. They are gram-negative straight rods, 0.3-1.0×1.0-6.0 □m, motile by peritrichous flagella, except for Tatumella, or nonmotile. They grow in the presence and absence of oxygen and grow well on peptone, meat extract, and (usually) MacConkey's media. Some grow on D-glucose as the sole source of carbon, whereas others require vitamins and/or mineral(s). They are chemoorganotrophic with respiratory and fermentative metabolism but are not halophilic. Acid and often visible gas is produced during fermentation of D-glucose, other carbohydrates, and polyhydroxyl alcohols. They are oxidase negative and, with the exception of Shigella dysenteriae 0 group 1 and Xenorhabdus nematophilus, catalase positive. Nitrate is reduced to nitrite except by some strains of Erwinia and Yersina. The G+C content of DNA is 38-60 mol % (Tₘ, Bd). DNAs from species from species within most genera are at least 20% related to one another and to Escherichia coli, the type species of the family. Notable exceptions are species of Yersina, Proteus, Providenica, Hafnia and Edwardsiella, whose DNAs are 10-20% related to those of species from other genera. Except for Erwinia chrysanthemi all species tested contain the enterobacterial common antigen (Bergy's Manual of Systematic Bacteriology, D. H. Bergy, et al., Baltimore: Williams and Wilkins, 1984).

E. coli are particularly useful as tyrosine overproducers however other enteric bacteria including Klebsiella, Salmonella, Shigella, Yersinia, and Erwinia may be converted to tyrosine over-producers and may then be used in the present invention. Additional examples of tyrosine over-producing strains that are suitable for the present method include, Microbacterium ammoniaphilum ATCC 10155, Corynebactrium lillium NRRL-B-2243, Brevibacterium divaricatum NRRL-B-2311, Arthrobacter citreus ATCC 11624, and Methylomonas SD-20. Other suitable tyrosine over-producers are known in the art, see for example Microbial production of L-tyrosine: A Review, T. K. Maiti et al, Hindustan Antibiotic Bulletin, vol 37, 51-65 (1995). Any strain that over-produces tyrosine may be used in preparing the enhanced tyrosine producing strains described herein.

### Prephenate Dehydrogenase

In the present invention an enzyme with tyrosine insensitive prephenate dehydrogenase activity is expressed in a tyrosine over-producing strain. Phenylalanine is still produced in tyrosine over-producing strains, even though pheA, which directs flow from prephenate into the phenylalanine pathway (FIG. 1), is inactive. It was found that increasing enzyme activity for directing prephenate into the tyrosine pathway was successful in increasing tyrosine production, and reducing production of phenylalanine. The prephenate dehydrogenase activity expressed is not feedback inhibited by tyrosine to allow maintenance of activity even when tyrosine is over-produced.

Expression in a tyrosine over-producing strain using any nucleic acid sequence encoding an enzyme with tyrosine insensitive prephenate dehydrogenase activity is suitable. For example, tyrosine insensitive prephenate dehydrogenases are present in members of the Bacterial Group III, where the classification is based on ribosomal RNA homology (Byng et al. (1980) J of Bacteriol 144:247-257). A cyclohexadienyl dehydrogenase is a type of prephenate dehydrogenase since the enzyme uses both prephenate and arogenate as substrates. In addition to converting prephenate to 4-OH-phenylpyruvate, which can be converted by an aminotransferase to tyrosine (see FIG. 1), a cyclohexadienyl dehydrogenase converts arogenate to tyrosine. Upon prephenate accumulation, arogenate may be formed by transamination of prephenate. However, with reduced prephenate accumulation due to the prephenate dehydrogenase activity, synthesis of arogenate will be less likely. Any enzyme with cyclohexadienyl dehydrogenase activity, where the prephenate dehydrogenase activity is not feedback inhibited by tyrosine, may be used in the present invention. The tyrc gene of Zymomonas mobilis encodes a cyclohexadienyl dehydrogenase that is tyrosine insensitive (Zhao et al. (1993) Eur J Biochem 212:157-65). The TyrC protein of Z. mobilis (SEQ ID NO:1) is particularly useful in the present invention.

DNA sequences from other organisms that encode enzymes having prephenate dehydrogenase activity, including cyclohexadienyl dehydrogenases, that are potentially tyrosine insensitive may be identified using the TyrC amino acid sequence (SEQ ID NO:1). For example, such sequences may be identified in members of the Bacterial Group III noted above, and in Rhodopseudomonas palustris, Rhodospirillum rubrum, and Agrobacterium tumefaciens as described in Example 6 herein. The TyrC amino acid sequence may be used in homology searching of protein databases such as with BLASTP as described in Example 6 herein, or of translations of DNA sequence databases such as with tBLASTn, as is well known to one skilled in the art.

In addition, the DNA sequence encoding Z. mobilis TyrC (SEQ ID NO:2; the natural GTG start was replaced with ATG for expression constructs) may be used to identify potentially tyrosine insensitive enzymes with prephenate dehydrogenase activity using methods well known to one skilled in the art. Examples of sequence-dependent protocols include, but are not limited to, methods of nucleic acid hybridization, and methods of DNA and RNA amplification as exemplified by various uses of nucleic acid amplification technologies [e.g., polymerase chain reaction (PCR), ligase chain reaction (LCR)].

For example, DNA sequences encoding enzymes with prephenate dehydrogenase activity could be isolated directly by using all or a portion of the known sequence as DNA hybridization probes to screen libraries from any desired plant, fungi, yeast, or bacteria using methodologies well known to those skilled in the art. Specific oligonucleotide probes based upon the literature nucleic acid sequences can be designed and synthesized by methods known in the art (Maniatis, supra). Moreover, the entire sequences can be used directly to synthesize DNA probes by methods known to the skilled artisan such as random primers DNA labeling, nick translation, or end-labeling techniques, or RNA probes using available in vitro transcription systems. In addition, specific primers can be designed and used to amplify a part of or full-length of the instant sequences. The resulting amplification products can be labeled directly during amplification reactions or labeled after amplification reactions, and used as probes to isolate full length cDNA or genomic fragments under conditions of appropriate stringency.

In addition, two short segments of the tyrC sequence may be used in polymerase chain reaction protocols, including RT-PCR, to amplify longer nucleic acid fragments encoding homologous genes from DNA or RNA. The polymerase chain reaction may also be performed on a library of cloned nucleic acid fragments wherein the sequence of one primer is derived from the tyrc sequence, and the sequence of the other primer takes advantage of the presence of the polyadenylic acid tracts to the 3′ end of the mRNA precursor encoding bacterial genes. Alternatively, the second primer sequence may be based upon sequences derived from the cloning vector. For example, the skilled artisan can follow the RACE protocol [Frohman et al., PNAS USA 85:8998 (1988)] to generate cDNAs by using PCR to amplify copies of the region between a single point in the transcript and the 3′ or 5′ end. Primers oriented in the 3′ and 5′ directions can be designed from the tyrc sequence. Using commercially available 3′ RACE or 5′ RACE systems (BRL), specific 3′ or 5′ cDNA fragments can be isolated [Ohara et al., PNAS USA 86:5673 (1989)]; and [Loh et al., Science 243:217, (1989)].

Any isolated nucleic acid molecule encoding an enzyme with prephenate dehydrogenase activity may be expressed in a tyrosine over-producing cell, typically as a component of a chimeric gene as described below herein, and the expressed enzyme may be assessed for tyrosine insensitivity based on enhancement of tyrosine production as described in Examples herein. In addition to identifying a naturally tyrosine insensitive prephenate dehydrogenase enzyme, a prephenate dehydrogenase that is not naturally tyrosine insensitive may be converted to tyrosine insensitivity by mutagenesis. Mutations may be made in the prephenate dehydrogenase coding region my methods well know to one skilled in the art, such as by error-prone PCR, and the resulting enzymes screened for tyrosine insensitivity. Any tyrosine insensitive prephenate dehydrogenase may be used in preparing an enhanced tyrosine over-producing strain.

Particularly suitable herein are nucleic acid molecules encoding enzymes having similarity to the Z. mobilis TyrC protein as set forth in SEQ ID NO:1. The skilled person will be able to use this sequence to find related sequences having tyrosine insensitive prephenate dehydrogenase activity by the methods described above. Accordingly it is contemplated that useful nucleic acid molecules will be selected from the group consisting of:
(a) an isolated nucleic acid molecule encoding the amino acid sequence as set forth in SEQ ID NO:1;
(b) an isolated nucleic acid molecule that hybridizes with (a) under the following hybridization conditions: 0.1×SSC, 0.1% SDS, 65° C. and washed with 2×SSC, 0.1% SDS followed by 0.1×SSC, 0.1% SDS; and
(c) an isolated nucleic acid molecule that encodes a polypeptide having 95% identity based on the Clustal method of alignment when compared to a polypeptide having the sequence as set forth in SEQ ID NO:1.

An amino acid sequence may have one or more substitutions of chemically equivalent amino acids, while maintaining the enzymatic activity. An isolated nucleic acid molecule encoding a tyrosine insensitive prephenate dehydrogenase may be codon optimized to provide optimal expression in a host of choice. An example of an isolated nucleic acid molecule sequence encoding TyrC, which may be used in an enhanced tyrpsine over-producing strain, is the natural coding sequence given as SEQ ID NO:2 (with the natural GTG start replaced with ATG for expression constructions).

### Recombinant Expression

An isolated nucleic acid molecule encoding a protein with tyrosine insensitive prephenate dehydrogenase enzyme activity for use in the present invention is operably linked to suitable regulatory sequences, typically in a chimeric gene construct, to allow expression in a recombinant host cell. Regulatory sequences include promoters and terminators for transcription, as well as translation control regions. Especially useful are regulatory sequences that direct high level expression of foreign proteins and that allow control of the timing of expression. Promoters used are constitutive or regulated promoters. Promoters which are useful to drive expression of the instant coding regions in the desired host cell are numerous and familiar to those skilled in the art, such as inducible promoters araB, rhaB, lac, tac, trc, T7, T5, tetracycline promoter, trp promoter, luxR promoter, tightly regulated synthetic promoters derived from lac/tac promoter, lnt/att-mediated gene inversion-controlled promoters, acid-inducible promoters, salt inducible promoters, pHCA inducible promoters, and heat/cold inducible promoters; or constitutive promoters such as IpdA, gyrA, ycgG, and fbp. Particularly suitable for use in the present invention are the araB and IpdA promoters. Termination control regions may also be derived from various genes native to the preferred hosts or that are functional in the preferred hosts. Optionally, a termination site may be unnecessary; however, it is most preferred if included.

A chimeric gene for expression of a tyrosine insensitive prephenate dehydrogenase is typically added to a vector that is used to make a recombinant host cell suitable for the present invention. Vectors useful for the transformation of suitable host cells are well known by one skilled in the art. Typically the vector additionally contains sequences allowing autonomous replication or chromosomal integration and a marker. Autonomous replicating vectors are typically plasmids used in cloning and transformation procedures, which then are maintained within a recombinant cell. Vectors may also be used which promote the integration of the chimeric gene encoding a tyrosine insensitive prephenate dehydrogenase into the host cell genome. Such vectors may be for either random or site-directed integration, or for homologous recombination. A vector may have features allowing single cross-over or double-crossover types of homologous recombination. Transformation of the vector into a host cell is by methods well know in the art such as uptake in calcium treated cells, electroporation, freeze-thaw uptake, heat shock, lipofection, electroporation, conjugation, fusion of protoplasts, and biolistic delivery.

The marker provides a trait for identifying transformed cells by methods including selection and screening. The marker is used to identify those cells that receive the transforming plasmid or integrated DNA. Types of usable markers include screening and selection markers. Many different selection markers available for recombinant cell selection may be used, including nutritional markers, antibiotic resistance markers, metabolic markers, and heavy metal tolerance markers. Some specific examples include, but are not limited to, thyA, serA, ampicillin resistance, kanamycin resistance, carbenicillin resistance, spectinomycin resistance, and mercury tolerance. In addition, a screenable marker may be used to identify recombinant cells. Examples of screenable markers include GFP, GUS, carotenoid production genes, and beta-galactosidase. A particularly suitable marker in the instant invention is a selectable marker.

### Production of Tyrosine

Enteric bacterial strains of the present invention that have enhanced tyrosine production make tyrosine that is excreted into the medium. These strains may be grown in a fermenter where commercial quantities of tyrosine are produced. For example, strain DPD4561, a tyrosine over-producing strain that expresses Zymomonas mobilis TyrC described in Example 3 herein, produced 59.1 g/L tyrosine in a fermentation.

Production fermentation or “scale up” fermentation in this disclosure describes greater than 10 L aerobic batch fermentation, and usually 200 L or greater. Where commercial production of tyrosine is desired, a variety of culture methodologies may be applied. For example, large-scale production from a recombinant microbial host may be produced by both batch and continuous culture methodologies. A classical batch culturing method is a closed system where the composition of the medium is set at the beginning of the culture and not subjected to artificial alterations during the culturing process. Thus, at the beginning of the culturing process the medium is inoculated with the desired organism or organisms and growth or metabolic activity is permitted to occur adding nothing to the system. Typically, however, a “batch” culture is batch with respect to the addition of carbon source and attempts are often made at controlling factors such as pH and oxygen concentration. In batch systems the metabolite and biomass compositions of the system change constantly up to the time the culture is terminated. Within batch cultures cells moderate through a static lag phase to a high growth log phase and finally to a stationary phase where growth rate is diminished or halted. If untreated, cells in the stationary phase will eventually die. Cells in log phase are often responsible for the bulk of production of end product or intermediate in some systems. Stationary or post-exponential phase production can be obtained in other systems.

A variation on the standard batch system is the Fed-Batch system. Fed-Batch culture processes are also suitable in the present invention and comprise a typical batch system with the exception that the substrate is added in increments as the culture progresses. Fed-Batch systems are useful when catabolite repression is apt to inhibit the metabolism of the cells and where it is desirable to have limited amounts of substrate in the medium. Measurement of the actual substrate concentration in Fed-Batch systems is difficult and is therefore estimated on the basis of the changes of measurable factors such as pH, dissolved oxygen (DO) and the partial pressure of waste gases such as CO₂. Batch and Fed-Batch culturing methods are common and well known in the art and examples may be found in Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol., 36, 227, (1992), herein incorporated by reference.

Fermentation media contain suitable carbon substrates. Suitable substrates may include but are not limited to monosaccharides such as glucose and fructose, oligosaccharides such as lactose or sucrose, polysaccharides such as starch or cellulose or mixtures thereof and unpurified mixtures from renewable feedstocks such as cheese whey permeate, cornsteep liquor, sugar beet molasses, and barley malt. The carbon substrates may also comprise, for example, alcohols, organic acids, proteins or hydrolyzed proteins, or amino acids. Hence, it is contemplated that the source of carbon utilized in the present fermentation may encompass a wide variety of carbon containing substrates.

Commercial production of tyrosine may also be accomplished with a continuous culture. Continuous cultures are open systems where a defined culture medium is added continuously to a bioreactor and an equal amount of conditioned medium is removed simultaneously for processing. Continuous cultures generally maintain the cells at a constant high liquid phase density where cells are primarily in log phase growth. Alternatively, continuous culture may be practiced with immobilized cells where carbon and nutrients are continuously added, and valuable products, by-products or waste products are continuously removed from the cell mass. Cell immobilization may be performed using a wide range of solid supports composed of natural and/or synthetic materials.

Continuous or semi-continuous culture allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by medium turbidity, is kept constant. Continuous systems strive to maintain steady state growth conditions and thus the cell loss due to medium being drawn off must be balanced against the cell growth rate in the culture. Methods of modulating nutrients and growth factors for continuous culture processes as well as techniques for maximizing the rate of product formation are well known in the art of industrial microbiology and a variety of methods are detailed by Brock, supra.

Particularly suitable for tyrosine production is a fermentation regime as follows. The desired strain that is converted to a tyrosine over-producing strain by the present method is grown in shake flasks in semi-complex medium at about 35° C. with shaking at about 300 rpm in orbital shakers and then transferred to a 10 L seed fermentor containing similar medium. The seed culture is grown in the seed fermentor under constant air sparging until OD₅₅₀ is between 10 and 25, when it is transferred to the production fermentor where the fermentation parameters are optimized for tyrosine production. Typical inoculum volumes transferred from the seed tank to the production tank range from 2.0-10% v/v. Typical fermentation medium contains minimal medium components such as potassium phosphate (1.0-3.0 g/l), sodium phosphate (0-2.0 g/l), ammonium sulfate (0-1.0 g/l), magnesium sulfate (0.3-5/0 g/l), a complex nitrogen source such as yeast extract or soy based products (0-10 g/l). Trace amounts of L-phenylalanine and trace elements are also added to the medium at all stages of the seed train for optimal growth of the strain. Carbon sources such as glucose (or sucrose) are continually added to the fermentation vessel on depletion of the initial batched carbon source (10-30 g/l) to maximize tyrosine rate and titer. Carbon source feed rates are adjusted dynamically to ensure that the culture is not accumulating glucose in excess, which could lead to build up of toxic byproducts such as acetic acid. In order to maximize yield of tyrosine produced from substrate utilized such as glucose, biomass growth is restricted by the amount of phosphate that is either batched initially or that is fed during the course of the fermentation. The fermentation is controlled at pH 6.8-7.2 using ammonium hydroxide and either sulfuric or phosphoric acid. The temperature of the fermentor is controlled at 32-35° C. and the DO is maintained around 10-25% air saturation by cascade control using agitation (rpm) and airflow (SLPM) as variables. In order to minimize foaming, antifoam agents (any class-silicone based, organic based etc) are added to the vessel as needed. A particularly suitable antifoam agent used is Biospumex153K. For maximal production of tyrosine, the culture may be induced with small concentrations of isopropyl-β-D-thiogalactopyranoside (IPTG) (0-1.0 mM) at OD₅₅₀ 8-10. An antibiotic, for which there is an antibiotic resistant marker in the strain, such as kanamycin, may be used optionally to minimize contamination.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### General Methods

Standard recombinant DNA and molecular cloning techniques used in the Examples are well known in the art and are described, “Maniatis” supra, Enquist supra; and by Ausubel supra.

Standard genetic methods for transduction used in the Examples are well known in the art and are described by Miller, J. H., Experiments in Molecular Genetics, Cold Spring Harbor Laboratory Press, Cold Spring Harbor, N.Y. (1972).

The meaning of abbreviations is as follows: “kb” means kilobase(s), “bp” means base pairs, “nt” means nucleotide(s), “hr” means hour(s), “min” means minute(s), “sec” means second(s), “d” means day(s), “L” means liter(s), “ml” means milliliter(s), “μL” means microliter(s), “μg” means microgram(s), “ng” means nanogram(s), “mM” means millimolar, “μM” means micromolar, “nm” means nanometer(s), “μmol” means micromole(s), “pmol” means picomole(s), “ppm” means parts per million, “vvm” means volume air per volume liquid per minute, “CFU” means colony forming unit(s), “NTG” means N-methyl-N′-nitro-N-nitrosoguanidine, “IPTG” means isopropyl β-D-thiogalactopyranoside, “phenylalanine” or “phe” means L-phenylalanine, and “tyrosine” or “tyr” means L-tyrosine. “TFA” is trifluoroacetic acid, “ACN” is acetonitrile, “Kan^{R}” is kanamycin resistant, “Amp^{R}” is ampicillin resistant, “Phe” is phenylalanine auxotrophic, “Cm” is chloramphenicol, “Kan” is kanamycin, “Tet” is tetracycline, “CIP” is calf intestinal alkaline phosphatase, “LR” is ligase chain reaction.

### Media and Culture Conditions:

Materials and methods suitable for the maintenance and growth of bacterial cultures were found in Experiments in Molecular Genetics (Jeffrey H. Miller), Cold Spring Harbor Laboratory Press, Cold Spring Harbor, N.Y. (1972); Manual of Methods for General Bacteriology (Phillip Gerhardt, R. G. E. Murray, Ralph N. Costilow, Eugene W. Nester, Willis A. Wood, Noel R. Krieg and G. Briggs Phillips, eds), pp. 210-213, American Society for Microbiology, Washington, D.C. (1981); or Thomas D. Brock in Biotechnology: A Textbook of Industrial Microbiology, Second Edition (1989) Sinauer Associates, Inc., Sunderland, Mass. All reagents and materials used for the growth and maintenance of bacterial cells were obtained from Aldrich Chemicals (Milwaukee, Wis.), BD Diagnostic Systems (Sparks, Md.), Invitrogen Corp. (Carlsbad, Calif.), or Sigma Chemical Company (St. Louis, Mo.) unless otherwise specified.

LB medium contains the following in gram per liter of medium: Bacto-tryptone (10), Bacto-yeast extract, (5.0), and NaCl, (10).

Vogel-Bonner medium contains the following in gram per liter: MgSO₄.7H₂O, (0.2); citric acid-1H₂O, (2.0), K₂HPO₄, (10); and NaNH₄HPO₄.4H₂O, (3.5).

SOB medium contains the following in gram per liter: Bacto-tryptone, (20), Bacto-yeast extract (5.0), and NaCl (0.5), 250 mM KCl (10 ml), pH adjusted to 7.0 with NaOH.

Above media were either autoclaved or filter-sterilized. Vitamin B1 (thiamin) was added at 0.0001% to Vogel-Bonner medium. MgCl₂ was added to SOB medium (5.0 ml of 2M solution per liter). Carbon source and other nutrients and supplements were added as mentioned in the Examples. All additions were pre-sterilized before they were added to the media.

10X MOPS based minimal medium was purchased from Teknova (Half Moon Bay, Calif.). The MOPS minimal medium was made as follows per liter: 10XMOPS (100 ml), 0.132 M K₂HPO₄ (10 ml), 20% Glucose (10 ml). Other supplements were added as mentioned in the Examples. All additions were pre-sterilized before they were added to the medium.

SOC medium was obtained from Invitrogen (Carlsbad, Calif.).

Bochner selection plates as modified by Maloy and Nunn (1981, J. Bacteriol. 145:1110-1112) were made as follows:

### Solution A

| | | |
|---|---|---|
| Bacto tryptone | 5.0 | g |
| Bacto yeast extract | 5.0 | g |
| Chlortetracycline | 50 | mg (4.0 ml of aqueous |
| | | 12.5 mg/ml, stored dark, 4° C.) |
| Agar | 15 | g |
| H₂O | 500 | ml |

### Solution B

| | | |
|---|---|---|
| NaCl | 10 | g |
| NaH₂PO₄•H₂O | 10 | g |
| H₂O | 500 | ml |

Solutions A and B were autoclaved separately for 20 minutes at 15 psi, then mixed and cooled to pouring temperature. 5.0 ml of 20 mM ZnCl₂ and 6.0 ml of 2 mg/ml fusaric acid were added prior to pouring plates.

### Molecular Biology Techniques:

Restriction enzyme digestions, ligations, transformations, and methods for agarose gel electrophoresis were performed as described in Maniatis. Polymerase Chain Reaction (PCR) techniques were found in White, B., PCR Protocols: Current Methods and Applications, Volume 15 (1993) Humana Press Inc, Totowa, N.J.

### HPLC Method

High performance liquid chromatography was performed on an Agilent 1100 (Agilent Technologies, Palo Alto, Calif.). A ZORBAX SB-C18 column (Agilent Technologies) was used. The method used required a column flow rate of 1.00 ml/min, with a stop time of 11 minutes and a post time of 5 minutes. The mobile phase was composed of 95% Solvent A (water+0.1% TFA) and 5% Solvent B (ACN+0.1% TFA). The pump ran within pressure limits defined as a minimum of 20 bar and a maximum of 400 bar. The spectrum was scanned from 100 nm to 380 nm, with signal for tyrosine being recorded at 225 nm and a retention time of 3.598 minutes. Phenylalanine was detected at 215 nm, with a retention time of 4.388 minutes.

### Prephenate Dehydrogenase Microtiter Plate Activity Assay

The prephenate dehydrogenase assay (J. Dayan and D. B. Sprinson, “Determination of Prephenate dehydrogenase activity”, in Methods in Enzymology 1970, vol. 17A, p 562-563, N. O. Kaplan, S. P. Colowick, editors) measures the 4-hydroxyphenylprephenate (4-HPP) formed when prephenate is used as substrate. The quantity of 4-HPP formed is determined against a standard curve generated from authentic standards of 4-HPP treated under identical conditions. The extinction coefficient of 4-HPP at 330 nm is 5000. The assay reactions for each sample were conducted in a 500 μL microcentrifuge tube. The following buffer and reagent solutions were prepared: a) 50 mM Tris, pH 8.0 buffer containing 1 mM EDTA, 1 mM DTE prepared fresh weekly and stored at 5° C.; b) 10 mM barium prephenate in the Tris buffer; c) 10 mM NAD in the Tris buffer; 15 mM 4-HPP solution in buffer; d) 15% v/v trichloroacetic acid solution; e) 2 M sodium arsenate, pH 6.5; f) 1 M boric acid in 2M sodium arsenate, pH 6.5; and g) the enzyme extract diluted with the Tris buffer such that when 40 μL is added to the assay mixture, no more than 20 percent conversion is achieved and the quantity of 4-HPP formed does not exceed the standard range. The tube contained the following: 40 μL barium prephenate solution; 40 μL NAD solution; 100 μL Tris buffer. The contents were mixed, set in a water bath and pre-warmed to 37° C. The reaction was initiated by adding 20 μL of cell free extract or a 4-HPP standard solution to bring the reaction volume to 200 μL. The contents were vigorously mixed by vortexing and were allowed to incubate for 30 minutes at 37° C. Immediately following the reaction, the enzymatic assay was quenched with 40 μL 15% trichloroacetic acid (TCA). After 10 minutes the tube was set on ice for 5 minutes then centrifuged (12,000 rpm, 3 minutes). Two 40 μL aliquots of the supernatant were removed and transferred to wells in adjacent rows of a 96 well plate with a UV-transparent bottom (Corning-Costar, Catalog #3635 UV microtiter plates). Sodium arsenate buffer (100 μL) was added to one row of samples and standards; sodium arsenate/boric acid buffer (100 μL) was added to the second duplicate row of samples and standards. The 96-well plate was mixed slowly for 1 minute prior to reading the optical density at 330 nm on a microtiter plate reader. The difference in optical density between the samples with and without boric acid was recorded. This difference is the hydroxyphenylprephenate complex and the values from the HPP standards were used to generate a standard curve and derive a relationship between OD₃₃₀ and HPP concentration. A typical set of 4-HPP standards and the corresponding values at OD₃₃₀ are shown in Table 1. The following equation was used to determine the 4-HPP formed in test samples.

PPDH activity was calculated as follows:

Total prephenate dehydrogenase activity (μmoles/min)=

CFE dilutions*[4-HPP]/30 min

### PPDH specific activity (U/g)=total PPDH activity (μmoles/min) divided by the amount of protein in the cfe preparation.

TABLE 1
Typical OD₃₃₀ values for 4-HPP standards

| volume 15 mM | | | | |
|---|---|---|---|---|
| 4-HPP | | | | |
| solution | [4-HPP] | OD₃₃₀ w/ | OD₃₃₀ w/o | |
| (mL) | (μmoles) | Borate | Borate | delta |
| 0 | 0.000 | 0.202 | 0.102 | 0.1 |
| 0.01 | 0.150 | 0.34 | 0.137 | 0.203 |
| 0.02 | 0.300 | 0.48 | 0.179 | 0.301 |
| 0.03 | 0.450 | 0.615 | 0.213 | 0.402 |
| 0.04 | 0.600 | 0.721 | 0.255 | 0.466 |
| 0.05 | 0.750 | 0.861 | 0.294 | 0.567 |

### Prephenate Dehydrogenase Western Blot Procedure to Determine Protein Expression.

Polyclonal antisera for prephenate dehydrogenases (TyrA and TyrC) were generated from purified protein preparations. The antisera were produced in rabbits by standard procedures. The final production bleeds were used as antisera and diluted 1000-fold to use in the Western blot protocol. Sample preparation: cell lysates were created by breaking the cells by French Press or by sonication in 50 mM Tris buffer, pH 8. The cell free extract was isolated after centrifugation at 12,000 rpm, for 15′, at 4° C. The cell free extracts were all standardized to protein concentration of 0.1 to 0.125 mg/mL by dilution in 50 mM Tris, pH 8. The samples were diluted in 4×LDS with 10% reducing agent (sample loading buffer, Invitrogen Cat. #NP0007), placed in microfuge tubes, capped and heat-treated at 70° C. for 10 minutes.

Polyacrylamide gel electrophoresis: The standards were loaded in the range of 0.5, 2.5, 5, 10 and 20 ng of total protein per lane. The samples were loaded onto NuPage 1.5 mm 4-12% Bis-Tris gels (In-vitrogen NP0322). MOPS buffer (NP0001) was preferred for high molecular weight resolution. The samples were loaded to achieve a 5 μg/lane protein load. The Multi-Mark™ molecular weight standards or the Invitrogen SeeBlue® Prestained (Catolog #LC5625) were loaded in lane 1 (10 μl of Invitrogen # LC5677). Gels were run at constant voltage (200 V) for 40-50 minutes. The gel was carefully disassembled, rinsed in deionized, distilled water for 5 minutes and transferred onto a Problott™ Polyvinyldifluoride (PVDF) membrane by electroblot using standard techniques. The PVDF membrane was pre-soaked in 100% methanol for 5 seconds, then soaked in the transfer buffer before assembly of the transfer cassette. A sandwich of sponges, cellulose backing paper, PVDF and gel was assembled and placed in an electroblot apparatus. The chilled transfer buffer was 10 mM CAPS, pH 11 buffer with 10% v/v methanol. The transfer cassette was run at constant voltage (60V) for approximately 75 minutes or overnight at 20 V to ensure complete transfer of protein in the gels onto the membrane. The transfer cassette was disassembled and the PVDF membrane was rinsed in deionized water, rinsed quickly in 100% methanol, then air dried completely for ˜30 minutes. To initiate the first antibody reaction, the PVDF membrane was placed in a small, deep tray containing 20 mL solution containing 20 μL prephenate dehydrogenase polyclonal antisera and 20 mL 1% w/v Bovine serum albumin (BSA), 0.5% w/v Tween 20 in phosphate buffered saline (0.1 M phosphate, 0.15 M sodium chloride, pH 7.2 (Pierce BupH PBS cat. # 28372)). The tray containing the membrane in the first antibody was gently rocked for 1 hour at room temperature or overnight at 4° C. The membrane was washed 3×60 seconds with 20 mL 1% w/v Bovine serum albumin (BSA), 0.5% w/v Tween: 20 in phosphate buffered saline (0.1 M phosphate, 0.15 M sodium chloride, pH 7.2 (Pierce BupH PBS cat. # 28372)). The membrane was transferred to a clean tray containing 10 μL goat anti-rabbit IgG˜Horse radish peroxidase labeled (Pierce catalog #31460) in 20 mL 1% w/v Bovine serum albumin (BSA), 0.5% w/v Tween 20 in phosphate buffered saline (0.1 M phosphate, 0.15 M sodium chloride, pH 7.2 (Pierce BupH PBS cat. # 28372)). The tray containing the blot in the first antibody was gently rocked for 1 hour at room temperature, then washed 4×60 sec with 20 mL 1% w/v Bovine serum albumin (BSA), 0.5% w/v Tween 20 in phosphate buffered saline (0.1 M phosphate, 0.15 M sodium chloride, pH 7.2 (Pierce BupH PBS cat. # 28372)). The prephenate dehydrogenase reactive bands were developed within 1 to 2 minutes following treatment of the membrane with a solution containing 20 mL Vector SG stain (Vector Industries, Catalog #SK-4700). Band development was stopped by a water rinse. The prephenate dehydrogenase band was quantitated with a FluorChem densitometry system. The band corresponding to the prephenate dehydrogenase was integrated and compared against a curve generated by prephenate dehydrogenase standards to determine the quantity of prephenate dehydrogenase in the cell free extract.

### Bacterial Strains

### DPD4009

E. coli strain DPD4009 was constructed as described in US 20050260724 A1, which is herein incorporated by reference. DPD4009 is a tyrosine-overproducing, plasmid-less, phenylalanine auxotroph, which was derived from E. coli TY1 (DGL430), a tyrosine overproducing strain obtained from OmniGene Bioproducts, Inc. (Cambridge, Mass.). First, TY1 was cured of the plasmid it was carrying to yield a tetracycline-sensitive strain called TS5. Subsequently, TS5 was the recipient in a P1-mediated transduction using E. coli strain CAG12158, which carries pheA18::Tn10 (Coli Genetics Stock Center, Yale University, #7421), as the donor. One tetracycline-resistant transductant was called BNT565.2. BNT565.2 was the recipient in a P1-mediated transduction using E. coli strain WS158 as the donor. WS158 carries Ptrc-tyrA [KanR], a chromosomal modification resulting in the strong trc promoter driving tyrA expression. The pheA and tyrA genes are tightly linked on the chromosome, so selection was made for rare transductants that were resistant to both tetracycline and kanamycin. One such transductant was called DPD4009, which was shown to require phenylalanine for growth and to excrete tyrosine. DPD4009 therefore has an inactivated pheA gene due to the Tn10 insertion, and the trc promoter regulating expression of tyrA. Details of the construction of this strain are found in US 20050/260724 (in particular General Methods), which is herein incorporated by reference.

### DPD4515

Tyrosine over-producing strain E. coli DPD4515 was constructed as described in US 20050260724 A1, by transformation of E. coli strain DPD4009 using plasmid pCL101 EA, which carries E. coli aroEACBL genes in pCL1920 (obtained from Central Bureau for Fungal Cultures, Baarn, The Netherlands), and selection for spectinomycin resistance. The pCL101 EA plasmid was constructed as described by Valle et al. in U.S. Patent Application Publication No. 2002/0155521 (in particular Example 7), which is incorporated herein by reference.

### DPD4083

DPD4083 is described in co-owned and copending U.S. patent application Ser. No. 11/448,331, incorporated herein by reference. A chromosomal region was constructed in this strain that includes a deletion of the pheA and pheL coding regions along with their promoter region, as well as replacement of the endogenous tyrA promoter with the trc promoter. The tetRA circle method was used to make a complete deletion of pheL, pheA, and the promoter driving their expression. Two 140mer PCR primers were designed having adjacent 60 nucleotide regions of homology for the each of the upstream (3′ end of yfiA and intergenic region upstream of the promoter; called A) and downstream (3′ end of tyrA and intergenic region; called B) chromosomal regions flanking the desired deletion. One of these 140mer primers (Primer ABTR; SEQ ID NO:3) also had at the 3′ end a 20 nucleotide region of homology for the tetR gene encoding the regulatory gene from the transposon Tn10. The other 140mer primer (Primer BATA; SEQ ID NO:4) also had at the 3′ end a 20 nucleotide region of homology for the tetA gene, encoding a tetracycline efflux antiporter that confers tetracycline resistance. In addition, 20mer primers with the same regions of homology to tetR (Primer TR; SEQ ID NO:5) or tetA (Primer TA: SEQ ID NO:6) as at the 3′ ends of the ABTR and BATA primers, respectively, were used. These primers were obtained from Sigma Genosys (The Woodlands, Tex.).

The template DNA for PCR reactions using these primers can be obtained from any strain carrying the tetR and tetA genes. It is convenient to use a strain with the transposable element Tn10 located anywhere in the chromosome, such as E. coli DPD2112 (zib615::Tn10) or S. typhimurium TT2385 (zii614::Tn10). Template DNA, 0.5 μL per PCR reaction, was prepared by resuspending a single colony of DPD4112 or TT2385 in 32.5 μL water and 7.5 μl DMSO and heating at 95° C. for 10 minutes. Two PCR reactions, 50 μL, were performed. For the first PCR reaction, primer TR and BATA were used (3 μL of each primer at 10 pmol/μL) with template DNA from TT2385. For the second PCR reaction, primers TA and ABTR were used (3 μL of each primer at 10 pmol/μL) with template DNA from DPD4112. Water, 18.5 μL and ExTaq Premix (TaKaRa Bio Inc. Otsu, Shiga, Japan), 25 μL, were added. The PCR reaction conditions were 94° C./5 min+35×(94° C./1 min; 60° C./2 min; 72° C./3 min)+72° C./15 min. Products of the expected size, 2151 bp, were generated and purified with Qiaquick PCR purification kit (Qiagen, Valencia, Calif.).

The PCR products were denatured and reannealed to form tetRA circles as follows. Approximately equimolar amounts of each PCR product were combined and NaCl was added to a final concentration of 150 mM. These were heated to 100° C., then cooled slowly over 1 hour to 4° C. in a thermocycler using the following conditions 100° C./5 minutes, 95° C./3 minutes, 18 additional cycles of 3 minutes each with a decrease in temperature of 5° C. each cycle 4° C./hold. The reactions were desalted using a Microcon spin filter with 30,000 MW cutoff (Millipore Corp., Bedford, Mass.). Sterile water was added to 500 μL total volume. The columns were spun at speed 12 in a microfuge for 10 minutes. Water was added, 500 μL, and the columns were spun again. Prior to the final spin, 200 μL water was added. If necessary, 25 μL of water was added to recover the sample. The tetRA circles are open circular molecules carrying the complete tetR and tetA genes and the regions flanking the desired deletion.

The desalted tetRA circles, 10 μL, were used in electroporation of E. coli K12 MG1655 (ATCC#700926). Electroporation competent cells were prepared from a room temperature, stationary overnight 35 mL culture in SOB without magnesium inoculated with a single colony. Cultures were incubated with shaking at 30° C. until the culture reached a reading of 50 on a Klett-Summerson colorimeter with a red filter. The cells were pelleted by centrifugation, 15 minutes, setting 9, 4° C., Sorvall RT6000B, then resuspended with 3.0 ml ice cold water, and transferred to microfuge tubes, which were spun for 30 seconds at 4° C., in a microfuge. Following four more ice cold water washes, the cells were resuspended with 150 μL ice cold water and 50 or 60 μL were used for each electroporation. The electroporation conditions were 0.1 mm cuvette, 25 μF, 1.85 kV, 200 ohms. Then 750 μL SOC was added, the culture transferred to a microfuge tube, and incubated for 4 hours at 30° C. or overnight at 30° C. The electroporated cells were plated on LB plates with 15-20 μg/mL tetracycline and incubated at 37° C. for 1-3 days. In order for colonies to be tetracycline resistant, the tetR and tetA genes must be integrated into the E. coli chromosome. This may occur through homologous recombination using the A region homology to the chromosome. Likewise, integration is also possible using the B region of homology.

Tetracycline resistant colonies, carrying the integrated tetA and tetR genes, were purified on LB plates with 15-20 μg/mL tetracycline. A second, non-selective purification was done by streaking from single colonies selected from the LB plate with tetracycline to LB plates lacking tetracycline. The counter-selection for tetracycline sensitive derivatives, which are resistant to fusaric acid, was done on Bochner selection plates as modified by Maloy and Nunn (1981, J. Bacteriol. 145:1110-1112). Single colonies from the LB plate were streaked to these tetracycline-sensitive selection plates that were incubated at 42° C. for 2 days. Tetracycline sensitive colonies from these plates were purified on LB plates and subsequently tested for growth on minimal plates with or without phenylalanine.

Using this method, 12-18% of the tetracycline-sensitive isolates (from originally tetracycline resistant lines) did not grow on minimal plates without phenylalanine. These phenylalanine auxotrophs were formed by a recombination that removed the tetR and tetA genes and the pheA and pheL coding regions as well as their promoter (B×B recombination). Due to the nature of this method, these phenylalanine auxotrophic strains carried a precise deletion of the pheA and pheL coding regions and their promoter (ΔpheLA). One such phenylalanine auxotrophic strain that was retained was named DPD4072.

The two PCR fragments integration method (PCT Int. Appl WO 2004056973 A2) was used to place the strong trc promoter in the chromosome of E. coli K12 such that it would drive expression of the tyrA gene. This method also results in a kanamycin resistance cassette with flanking flp sites located immediately adjacent to the trc promoter (Ptrc).

A first linear DNA fragment (1581 bp) containing a kanamycin selectable marker flanked by site-specific recombinase target sequences (FRT) was synthesized by PCR using the kanamycin resistance gene of plasmid pKD4 (Datsenko and Wanner, PNAS, 97:6640-6645 (2000)) as a template. The primer pairs used were, T-kan(tyrA) (SEQ ID NO:7: 5′-AATTCATCAGGATCTGAACGGGCAGCTGACGGCTCGCGTGGCTTAAC GTCTTGAGCGATTGTGTAG-3′) which contains a homology arm (underlined, 46 bp) chosen to match sequences in the upstream region of the aroF stop codon, which is upstream of the tyrA gene in the E. coli chromosome, and a priming sequence for the kanamycin resistance gene (20 bp) and B-kan(trc) (SEQ ID NO:8: 5′-AAAACATTATCCAGAACGGGAGTGCGCCTTGAGCGACACGAATATGA ATATCCTCCTTAGTTCC-3′) that contains a homology arm (underlined, 42 bp) chosen to match sequences in the 5′-end region of the trc promoter DNA fragment and a priming sequence for the kanamycin resistance gene (22 bp). A second linear DNA fragment (163 bp) containing a trc promoter comprised of the −10 and −35 consensus sequences, lac operator (lacO), and ribosomal binding site (rbs) was synthesized by PCR from plasmid pTrc99A (Invitrogen, Carlsbad, Calif.) with primer pairs, T-trc(kan) (SEQ ID NO:9: 5′-CTAAGGAGGATATTCATATTCGTGTCGCTCAAGGCGCACT-3′) that contains a homology arm (underlined, 18 bp) chosen to match sequences in the downstream region of the kan open reading frame and a priming sequence for the trc promoter (22 bp) and B-trc(tyrA) (SEQ ID NO:10: 5′-CGACTTCATCAATTTGATCGCGTAATGCGGTCAATTCAGCAACCATG GTCTGTTTCCTGTGTGAAA-3′) that contains a homology arm (underlined, 46 bp) chosen to match sequences in the downstream region of the tyrA start codon and a priming sequence for the trc promoter (20 bp). The underlined sequences illustrate each respective homology arm, while the remainder are the priming sequences for hybridization to complementary nucleotide sequences on the template DNA for the PCR reaction. Standard PCR conditions were used to amplify the linear DNA fragments with MasterAmp™ Extra-Long DNA polymerase (Epicentre, Madison, Wis.) as follows;

| PCR reaction: | PCR reaction mixture: |
|---|---|
| Step1 94° C. 3 min | 1 μL plasmid DNA |
| Step2 93° C. 30 sec | 25 μL 2X PCR buffer #1 |
| Step3 55° C. 1 min | 1 μL 5′-primer (20 μM) |
| Step4 72° C. 3 min | 1 μL 3′-primer (20 μM) |
| Step5 Go To Step2, 25 cycles | 0.5 μL MasterAmp ™ DNA |
| | polymerase |
| Step6 72° C. 5 min | 21.5 μL sterilized dH₂O |

After completing the PCR reactions, PCR products were purified using Mini-elute QIAquick Gel Extraction Kit™ (QIAGEN Inc. Valencia, Calif.). The DNA was eluted with 10 μL of distilled water by spinning at top speed two times. The concentration of PCR DNA sample was about 0.5-1.0 μg/μL.

E. coli MC1061 strain carrying a λ-Red recombinase expression plasmid was used as a host strain for the recombination of PCR fragments. The strain was constructed by transformation with a λ-Red recombinase expression plasmid, pKD46 (amp^{R}) (Datsenko and Wanner, supra) into the E. coli strain MC1061. The λ-Red recombinase in pKD46 is comprised of three genes: exo, bet, and gam, expressed under the control of an arabinose-inducible promoter. Transformants were selected on 100 μg/mL ampicillin LB plates at 30° C. The electro-competent cells of E. coli MC1061 strain carrying pKD46 were prepared as follows. E. coli MC1061 cells carrying pKD46 were grown in SOB medium with 100 μg/mL ampicillin and 1 mM L-arabinose at 30° C. to an OD₆₀₀ of 0.5, followed by chilling on ice for 20 min. Bacterial cells were centrifuged at 4,500 rpm using a Sorvall® RT7 PLUS (Kendro Laboratory Products, Newton, Conn.) for 10 min at 4° C. After decanting the supernatant, the pellet was resuspended in ice-cold water and centrifuged again. This was repeated twice and the cell pellet was resuspended in 1/100 volume of ice-cold 10% glycerol.

Both the kanamycin marker PCR products (˜1.0 μg) and trc promoter PCR products (˜1.0 μg) were mixed with 50 μL of the competent cells and pipetted into a pre-cooled electroporation cuvette (0.1 cm) on ice. Electroporation was performed by using a Bio-Rad Gene Pulser set at 1.8 kV, 25 μF with the pulse controller set at 200 ohms. SOC medium (1.0 mL) was added after electroporation. The cells were incubated at 37° C. for 1.0 hour. Approximately one-half of the cells were spread on LB plates containing 25 μg/mL kanamycin. After incubating the plate at 37° C. overnight, six kanamycin resistant transformants were selected. The chromosomal integration of both the kanamycin selectable marker and the trc promoter in the front of the tyrA gene was confirmed by PCR analysis. A colony of transformants was resuspended in 25 μL of PCR reaction mixture containing 23 μL SuperMix (Invitrogen), 1.0 μL of 5′-primer T-ty(test) (SEQ ID NO:11: 5′-CAACCGCGCAGTGAAATGAAATACGG-3′) and 1.0 μL of 3′-primer B-ty(test) (SEQ ID NO:12: 5′-GCGCTCCGGAACATAAATAGGCAGTC-3′). Test primers were chosen to amplify regions located in the vicinity of the integration region. The PCR analysis with T-ty(test) and B-ty(test) primer pair revealed the expected size product of 1,928 bp on a 1.0% agarose gel. The resultant recombinant with Ptrc-tyrA::KanR was called E. coli WS158.

Generalized transduction using P1clr100Cm phage (J. Miller. Experiments in Molecular Genetics. 1972. Cold Spring Harbor Press) was used to combine the Ptrc-tyrA::Kan^{R} with ΔpheLA. Phage grown on E. coli strain WS158 carrying Ptrc-tyrA::Kan^{R} was used as the donor, E. coli strain DPD4072 with ΔpheLA was the recipient, and selection was for kanamycin resistance on LB plates with 12.5 μg/mL kanamycin. The transductants selected on 12.5 μg/mL were subsequently able to grow on plates containing 25 μg/mL kanamycin. The Kan^{R} transductant colonies were screened for phenylalanine auxotrophy by testing for growth on minimal medium with and without phenylalanine. Of 313 Kan^{R} colonies obtained, 4 required phenylalanine for growth. The presence of the Ptrc-tyrA::Kan^{R} in these 4 strains was confirmed by PCR amplifications. Thus, the observed cotransduction frequency of pheA and tyrA was >98%, as expected for adjacent genes. The Kan^{R}, Phe⁻ strains, each of which was a P1clr100Cm Iysogen, were retained and named DPD4081, DPD4082, DPD4083, and DPD4084.

### DPD4130

A non-K12 E. coli strain was that excretes a low level of phenylalanine (U.S. Pat. No. 2,973,304) was obtained from the ATCC (ATCC#13281) and renamed DPD4130. Strain DPD4130 requires tyrosine for growth.

### DPD4119

This tyrosine over-producing strain was converted from a phenylalanine over-producing strain. A high-level phenylalanine excreting strain was obtained in several steps. E. coli strain DPD4130 was subjected to mutagenesis using NTG followed by selection for analogue resistance using 3-fluorotyrosine. The resultant 3-fluorotyrosine resistant strain was mutagenized with NTG and selected for resistance to the analogue para-fluorophenylalanine. The resultant 3-fluorotyrosine and para-fluorophenylalanine resistant strain was mutagenized with NTG and selected for resistance to the analogue β-2-thienylalanine. The resultant 3-fluorotyrosine, para-fluorophenylalanine and β-2-thienylalanine resistant strain was mutagenized with NTG and a tyrosine auxotroph (Tyr⁻) was isolated. A tyrosine resistant mutant of the 3-fluorotyrosine, para-fluorophenylalanine, β-2-thienylalanine resistant and Tyr⁻ strain was selected. The resultant tyrosine, 3-fluorotyrosine, para-fluorophenylalanine, β-2-thienylalanine resistant and Tyr⁻ strain was selected for resistance to phage P1, Type I phage, and Type II phage and then a xylose negative mutant was isolated. The resultant strain was transformed with the plasmid pJN307, encoding a feedback resistant pheA gene with a deleted attenuator (described in Nelms et al. Appl Environ Microbiol. 1992 58(8):2592-8 and in U.S. Pat. No. 5,120,837). Finally a Tyr⁺ prototrophy was isolated and then a strain resistant to high phenylalanine and high temperature was obtained and named DPD4003. E. coli strain DPD4003 produces >40 g/l phenylalanine from glucose in fermentation.

The phenylalanine excreting strain DPD4003 was resistant to phage P1. Thus, a phage P1-sensitive revertant was isolated so that P1 mediated generalized transduction could be used to introduce new genetic material. E. coli DPD4003 was infected with P1clr100Cm and the rare Cm^{R} colonies were isolated. These spontaneous, putative P1-resistant revertants were then selected for growth at 42° C. to cure the temperature-sensitive lysogenic phage. One of these temperature-resistant and Cm-sensitive isolates, designated DPD4110, was confirmed to be sensitive to P1. This confirmation was done by testing the frequency of Cm^{R} colonies after infection by P1clr100Cm. A similar number of Cm^{R} colonies were obtained for infection of DPD4110 as were obtained for infection of DPD4130, which is the original parental strain of DPD4003.

E. coli strain DPD4110 contains plasmid pJN307, which carries a kanamycin resistance gene. Thus, a derivative of DPD4110 lacking this small plasmid was isolated. This was accomplished by treating DPD4110 with sub-lethal concentrations (50 or 75 μg/ml) of novobiocin for 22 hours in LB medium at 37° C. Single colonies from these cultures were tested for kanamycin-sensitivity and two such derivatives were retained. The loss of plasmid DNA in the Kan^{S} strains, DPD4112 and DPD4113, was confirmed by agarose gel electrophoresis of total DNA.

Strain DPD4112 was used as a recipient in a generalized transduction using a P1clr100Cm lysate of E. coli DPD4083, an E. coli K12 strain that carries the ΔpheLA Ptrc-tyrA::Kan^{R} chromosomal region. A high concentration of donor P1 phage, 100 μl of the phage lysate, and very low concentration of kanamycin, 3.0 μg/ml, were used. Spontaneous low-level kanamycin-resistant colonies, which were unable to grow in the presence of 25 μg/ml, kanamycin, occurred in this procedure. One colony that subsequently grew on high level, 25 μg/ml, kanamycin was obtained. This transductant was shown to be a phenylalanine auxotroph and to ferment sucrose, as expected for a strain resulting from transduction of the ΔpheLA Ptrc-tyrA::Kan^{R} chromosomal region into the recipient DPD4112. This new strain, DPD4118, was also a P1clr100 Iysogen, as indicated by its resistance to chloramphenicol and its temperature sensitivity. Thus, selection for growth at 42° C. was done. Two resultant strains, DPD4119 and DPD4120, were each shown to be chloramphenicol sensitive and to excrete tyrosine in a plate assay for cross feeding of the tyrosine auxotrophic E. coli strain, AT2471 (CGSC #4510).

### Example 1

### Cloning and Demonstration of Functional Expression from an araB Promoter of Zymomonas mobilis tyrC and Escherichia coli tyrA

This example describes the molecular cloning of Zymomonas mobilis tyrC and Escherichia coli tyrA genes. Furthermore, this example describes functional expression of tyrc and tyrA by complementation of a tyrA mutant of E. coli

The Gateway® (Invitrogen) cloning technology was used to make constructs for expression of the E. coli tyrA and Z. mobilis tyrC coding regions. Directional TOPO cloning was performed first to place these tyrA and tyrc coding regions into the entry vector, pENTR/SD/D-TOPO, using primers designed to amplify the codign regions and facilitate directional cloning. These primers were as follows:

### Forward primer for Z. mobilis tyrc includes a 5′ tail CACCTGA (SEQ ID NO:13): CACCTGATGACCGTCTTTAAGCATATT

### Reverse primer for Z. mobilis tyrC includes a stop codon but no tail (SEQ ID NO:14): TTAAGGGCGAATATCGTGGT

### Forward primer for E. coli K12 tyrA with 5′ tail CACCTG (SEQ ID NO:15): CACCTGATGGTTGCTGAATTGACCGC

### Reverse primer for E. coli K12 tyrA includes a stop codon but no tail (SEQ ID NO:16): TTACTGGCGATTGTCATTCG

PCR products were produced using the forward and reverse primers for tyrC with Z. mobilis genomic DNA as a template, and using the forward and reverse primers for tyrA with E. coli genomic DNA as template, by the standard protocol described in General Methods. The resulting products were cloned into the pENTR/SD/D-TOPO vector according to the manufacturer's instructions. After a 5 minute incubation of PCR product and vector, the reaction mixture was transformed into Top10 competent cells and plated on LB agar plates with 50 μg/ml kanamycin. Once transformants were obtained, single colonies were selected for mini-prep analysis. Isolated plasmids were digested with AscI and NotI to verify the insert. The insert DNA in several clones was verified by DNA sequence analysis. Two plasmids were used for further experiments, pENTRtyrA-3 (with the E. coli tyrA coding region) and pENTRtyrC1-1 (with the Z. mobilis tyrc coding region).

An LR reaction was performed according to the manufacturer's instructions to place the cloned coding regions into destination vectors. Each of the entry clones, pENTRtyrA-3 and pENTRtyrC1-1, was mixed with the destination vector, pBAD-DEST49, in the presence of LR Clonase enzyme. After a 60 minute incubation, the constructs were transformed into TOP10 cells and plated on LB agar plates with 100 μg/ml ampicillin. Several Amp^{R} single colonies were selected, plasmid DNA was isolated and restriction digestion with KpnI and HindIII was done to verify the inserts. A tyrA and a tyrc expression plasmid that were positive in the restriction analysis were named pBADtyrA-3 and pBADtyrC1-1, respectively. In these plasmids the tyrA and tyrc coding regions are inserted downstream of the araB promoter.

These two plasmids were then transformed into a tyrA⁻ E. coli strain, AT2471 (CGSC #4510; Taylor and Trotter (1967) Bacteriol. Rev. 31:332). Growth at 37° C. was tested on M9 minimal medium with glucose as a carbon source and thiamin addition. Presence of either pBADtyrA-3 or pBADtyrC1-1 in AT2471 allowed growth in the absence of tyrosine indicating functional expression of prephenate dehydrogenase activity.

### Example 2

### Improved Tyrosine Production with Expression of tyrC from an araB Promoter in an E. coli K12 Tyrosine Producing Strain

This example describes improved tyrosine production in an E. coli K12-derived tyrosine producing strain that carries the tyrc expression plasmid, pBADtyrC1-1. Furthermore, the titer of phenylalanine, was reduced.

An E. coli K12 derived tyrosine producing strain, DPD4515 (described in General Methods), was transformed separately with the plasmids pBADtyrA-3 and pBADtyrC1-1 constructed in Example 1, to give the strains DPD4554 (pBADtyrA-3 in DPD4515) and DPD4556 (pBADtyrC1-1 in DPD4515).

Tyrosine production was tested in shake flasks using MOPS buffered minimal medium with 2 g/l glucose and initial 10 μg/ml phenylalanine with varying concentrations of L-arabinose between 0.002% and 0.2%. Following incubation of strains DPD4554, DPD4556, and DPD4515 for 22 hours at 37° C., glucose was depleted and tyrosine and phenylalanine were measured by HPLC as described in General Methods. The results are shown in Table 2.

TABLE 2
Effects of tyrA and tyrC expression on tyrosine and phenylalanine levels.

| Strain arabinose | Arabinose | | |
|---|---|---|---|
| concentration | concentration | Ave. Tyr | Ave. Phe |
| DPD4554 | 0.2% | 374 | 45 |
| DPD4554 | 0.02% | 321 | 36 |
| DPD4554 | 0.002% | 316 | 35 |
| DPD4556 | 0.2% | 378 | 35 |
| DPD4556 | 0.02% | 372 | 31 |
| DPD4556 | 0.002% | 374 | 31 |
| DPD4515 | 0.2% | 280 | 88 |
| DPD4515 | 0.02% | 258 | 72 |

### Thus, expression of either E. coli tyrA (in strain DPD4554) or Z. mobilis tyrC (in strain DPD4556) improved tyrosine production and reduced phenylalanine production as compared with the control strain.

The strain expressing Z. mobilis tyrC yielded consistently higher tyrosine titers and reduced phenylalanine titers at all concentrations of arabinose, the inducer of the araB promoter expressing tyrc. The addition of arabinose had little effect on tyrosine production likely because of catabolite repression of the araB promoter as well as the Ara⁺ phenotype of these strains, which means that arabinose is catabolized. Accordingly, the araB promoter functions only at the basal level of expression in glucose medium.

The expression levels of tyrA and tyrc from the araB plasmids were measured by Western blots and prephenate dehydrogenase activity assays, in the E. coli K12 strains DPD4515 (K12 tyrosine producing strain), DPD4554 (pBADtyrA-3 in DPD4515) and DPD4556 (pBADtyrC1-1 and DPD4515). Shake flask experiments were done using a MOPS buffered medium with 2 g/l glucose and 10 μg/ml phenylalanine with and without addition of 0.2% arabinose, with incubation at 37° C. for 22 hours. HPLC was used to determine tyrosine and phenylalanine concentration, expression was measured on Western blots, and prephenate dehydrogenase activity was quantitated with an enzyme assay, all as described in General Methods. The results are summarized in Table 3.

TABLE 3
TyrA expression, prephenate dehydrogenase activity, tyrosine
and phenylalanine levels in tyrA and tyrC strains.

| | | | | | TyrA | PD |
|---|---|---|---|---|---|---|
| | | | Shake flask 2 g/l | | Western | activity |
| | | | glucose | | Blot | 15′ data |
| E. coli K12 | | | Tyrosine, | | % Total | umol/min/ |
| strain | Relevant Genotype | Ara | ppm | Phe, ppm | protein | mg × 10e2 |
| DPD4515 | pCL101EA (aroEACBL)/Ptrc-tyrA, | − | 256 | 76 | 0.25 | 2.6 |
| | pheA::Tn10, aroGfbr, tyrR | | | | | |
| | | + | 256 | 78 | 0.28 | 4.4 |
| DPD4554 | pBADtyrA-3/DPD4515 | − | 296 | 89 | 0.42 | 7 |
| | | + | 308 | 86 | 0.32 | 7.8 |
| DPD5446 | pBADtyrC1-1/DPD4515 | − | 348 | 72 | 0.29 | 21 |
| | | + | 374 | 70 | 0.23 | 12.7 |

The previous results that demonstrated improved tyrosine production and decreased phenylalanine in the strains with multicopy tyrA or tyrC were replicated in this experiment. Also, as in the previous results, arabinose did not substantially improve tyrosine production, and expression of tyrc had a greater effect than expression of tyrA. The Western Blot data indicated that the level of tyrA expression with addition of pBADtyrA-3 to DPD4515 was less than 2-fold increased over the chromosomal expression level in the control. Likewise, the prephenate dehydrogenase activity levels were only slightly increased with addition of pBADtyrA-3 to DPD4515. The Western blot showed no increase in the level of TyrA protein with addition of pBADtyrC-1. However, the prephenate dehydrogenase activity was clearly elevated in the strain carrying pBADtyrC-1. Although the prephenate dehydrogenase assay was somewhat variable, the trend of increased prephenate dehydrogenase activity with increased tyrosine titer was evident. These results provide evidence that the limitation in strains with the Ptrc-tyrA chromosomal construct, such as these E. coli K12 strains, is at flux though prephenate dehydrogenase. Accordingly, further improvement of prephenate dehydrogenase expression may further boost tyrosine production.

### Example 3

### Improved Tyrosine Production with Expression of Z. mobilis tyrC from an araB Promoter in a non-K12 Tyrosine Producing Strain

This example describes improved tyrosine production in a non-K12 E. coli tyrosine producing strain that carries the tyrC expression plasmid, pBADtyrC1-1.

The expression plasmids pBADtyrA-3 and pBADtyrC1-1, constructed in Example 1, were transformed into a high level tyrosine producing strain, DPD4119 (described in General Methods). The plasmids in the resulting strains were verified with restriction enzyme analysis and the strains were named DPD4560 (DPD4119 with pBADtyrA-3) and DPD4561 (DPD4119 with pBADtyrC1-1)

Stains DPD4119, DPD4560 and DPD4561 were tested in shake flask analysis in MOPS minimal medium with 2 g/l glucose and initial 10 μg/ml phenylalanine with either 0, 0.02%, and 0.2% of arabinose. Strain DPD4119 was used as a control. Cultures were incubated at 32° C. for 22 hours at which time the glucose was depleted. Results of HPLC analysis (as in General Methods) are given in Table 4. Addition of arabinose did not cause a significant change in gene expression, as measured by the concentration of tyrosine. This was likely due to catabolite repression of the araB promoter. Nonetheless, expression of either E. coli tyrA (in strain. DPD4560) or Z. mobilis tyrC (in strain DPD4561) improved tyrosine production as compared with the control strain, DPD4119. The strain expressing Z. mobilis tyrC yielded consistently higher tyrosine titers, and lower levels of phenylalanine.

TABLE 4
Enhanced tyrosine production with TyrC expression in non K-12 strain.

| STRAIN | | AVG TYR | AVG PHE |
|---|---|---|---|
| NAME | ARABINOSE, % | (PPM) | (PPM) |
| DPD4560 | 0 | 409 | 96 |
| DPD4560 | 0.2 | 413 | 95 |
| DPD4560 | 0.02 | 411 | 99 |
| DPD4561 | 0 | 446 | 84 |
| DPD4561 | 0.2 | 456 | 87 |
| DPD4561 | 0.02 | 453 | 83 |
| DPD4119 | 0 | 325 | 109 |
| DPD4119 | 0.2 | 327 | 104 |
| DPD4119 | 0.02 | 322 | 102 |

Shake flask studies were repeated with DPD4560, DPD4561 and DPD4119 in MOPS minimal medium with 2 g/l glucose and initial 10 μg/ml phenylalanine with 0.02% arabinose. These cultures were incubated at 32° C. for 22 hours at which time the glucose was depleted. The similar OD₆₀₀ values for each strain (Table 5) indicated that all strains grew to the same density. The results of HPLC analysis given in Table 5 demonstrate that multicopy expression of either tyrA or tyrc from the araB promoter resulted in 35% increase in tyrosine titer as well as reduction in the amount of phenylalanine produced. The reduction in the phenylalanine titer was greater for the strain carrying the Z. mobilis tyrc expression plasmid.

TABLE 5
Enhanced tyrosine production and normal growth with TyrC
expression in non-K12 strain.

| Strain name | Avg Tyr (ppm) | AvgPhe (ppm) | OD600 |
|---|---|---|---|
| DPD4119 | 326 | 106 | 1.14 |
| DPD4560 | 446 | 71 | 1.14 |
| DPD4561 | 450 | 57 | 1.13 |

### Example 4

### Improved Tyrosine Production with Expression of tyrC from an IpdA Promoter in an E. coli K12 Tyrosine Producing Strain

This example describes expression of Z. mobilis tyrc driven by the E. coli IpdA promoter, a strong promoter that does not require induction, resulting in improved tyrosine production.

Plasmid pDEW694 is part of the LuxArray of plasmids carrying E. coli promoters fused to the luxCDABE operon (Van Dyk et al. (2001) J. Bacteriol. 183: 5496-5505). pDEW694 has the strong promoter immediately upstream of IpdA driving luxCDABE expression. To use the IpdA promoter for driving gene expression, pDEW694 was converted to a Destination plasmid compatible with the Gateway® system in a process that concomitantly removed most of the luxCDABE operon. Plasmid pDEW694 was digested with Hind III and the resulting large DNA fragment was isolated from and agarose gel. The overhanging ends were filled with Klenow fragment of DNA polymerase to make them blunt, the fragment was treated with calf intestinal alkaline phosphatase and then ligated with the RfC.1 conversion cassette from Invitrogen. Following transformation of E. coli ccdB survival cells, and selection for chloramphenicol resistance, a transformant was obtained. Plasmid DNA isolated from this transformant was digested with Sma I to demonstrate that the Rfc.1 cassette was in the desired orientation, such that the LR reaction can be used to insert genes for expression from the IpdA promoter. This new Destination (DEST) vector was named pDEW697.

An LR reaction was performed according to the manufacturer's instructions to place the E. coli tyrA and Z. mobilis tyrC coding regions into the pDEW697 destination vector. The entry clones, pENTRtyrA-3 and pENTRtyrC1-1 (described in Example 1), were mixed with pDEW697 in the presence of LR Clonase enzyme. After a 60 minute incubation, the constructs were transformed into TOP10 cells and plated on LB agar plates with 100 μg/ml ampicillin. The new expression plasmids were named pDEW814 (E. coli tyrA coding region in DEST plasmid pDEW697 with IpdA promoter) and pDEW815 (Z. mobilis tyrC coding region in DEST plasmid pDEW697 with IpdA promoter).

Plasmids pDEW814 and pDEW815 were transformed into tyrosine producing E. coli K12 strain, DPD4515 (see General Methods), to yield strains DPD4598 and DPD4599, respectively. Shake flask experiments in MOPS buffered medium with 2 g/l glucose and initial 10 μg/ml phenylalanine were conducted with triplicate biological replicates at 37° C. for 22 hours. The data from HPLC analysis is given in the Table 6.

TABLE 6
Increased tyrosine using tyrC expressed from lpdA promoter.

| | | | Phenylalanine, | |
|---|---|---|---|---|
| Strain | Tyrosine, ppm | SD | ppm | SD |
| DPD4515 | 113 | 3 | 37 | 4 |
| DPD4598 | 134 | 22 | 41 | 0 |
| (PlpdA-tyrA) | | | | |
| DPD4599 | 186 | 2 | 24 | 1 |
| (PlpdA-tyrC) | | | | |

SD = standard deviation

These data demonstrated that the tyrosine titer was elevated by 65% with the tyrC expression plasmid. Furthermore, phenylalanine, was decreased by 30%.

### Example 5

### Comparison of Improvements in Tyrosine Production with Expression of tyrC or tyrA Mutants from an IpdA Promoter in an E. coli K12 Tyrosine Producing Strain

This example compares increased tyrosine production with expression of TyrC to effects on tyrosine production of tyrA mutants that are likely resistant to inhibition by tyrosine.

Feed-back resistant mutants of E. coli tyrA (tyrAfbr) that are not inhibited by tyrosine have been isolated (Lutke-Eversloh and Stephanopoulos (2005) Appl Environ Microbiol. 71:7224-7228). Each of these tyrAfbr mutants had multiple amino acid changes. Site directed mutants in tyrA carrying subsets of the mutations in tyrA described by Lutke-Eversloh and Stephanopoulos were constructed. Eight tyrA mutants were made starting with the E. coli K12 wild type tyrA coding region (SEQ ID NO:17), and produced the amino acid changes in the TyrA protein sequence (SEQ ID NO:18) at specified positions as listed in Table 7. These mutants were made using the QuikChange® II Site-Directed Mutagenesis Kit (Stratagene, La Jolla, Calif., Catalog #200524) according to the manufacturer's directions. This kit uses PfuUltra™ high-fidelity DNA polymerase to extend two oligonucleotide primers containing the desired mutation during thermal cycling. The primer is not displaced and the unmethylated product generated is treated with Dpn I to digest the original methylated DNA template. The mutated DNA is then transformed into XL1-Blue super-competent cells and plated for antibiotic selection. Because this kit is designed to construct single point mutations, the double and triple mutants were made in successive rounds. The wild-type tyrA gene was initially sequenced and found to contain a PCR induced point mutation, which changed an amino acid (51) in the tyrA gene from alanine (codon: GCA) to threonine (codon: ACA). Therefore, the altered base (G) was also changed to match the wild-type sequence (C). Each mutant was made using the appropriate complementary primer sets found in Table 7. These primers were designed following the manufacturer's specifications and HPLC purified by Sigma-Genosys (The Woodlands, Tex., USA).

TABLE 7

Primers and mutations made in tyrA, with the mutant codon
sequence is in bold.

| | | | | |
|---|---|---|---|---|
| | | | aa | |
| TyrA | Wild type | Mutant | Muta- | |
| Muta- | aa + | aa + | tion | Site-directed Mutagenesis primers |
| tion | sequence | sequence | site | 5′ to 3′ |

| | | | | |
|---|---|---|---|---|
| M53I | Methio- | Isoleu- | 53 | SEQ ID NO: 19 M53I-F = |
| | nine | cine | | GAGCGCGAGGCATCTATTTTGGCCTCGCG |
| | ATG | AATT | | SEQ ID NO: 20 M53I-R = |
| | | | | CGCGAGGCCAAAATAGATGCCTCGCGCTC |
| Q124R | Gluta- | Argi- | 124 | SEQ ID NO: 21 Q124R-F = |
| | mine | nine | | CCCTCTCGGGTTATCGGGTGCGGATTCTG |
| | CAG | CGG | | SEQ ID NO: 22 Q124R-R = |
| | | | | CAGAATCCGCACCCGATAACCCGAGAGGG |
| Y263H | Tyro- | Hista- | 263 | SEQ ID NO: 23 Y263H-F = |
| | sine | dine | | GCTACTTTTGCTCACGGGCTGCACCTGGC |
| | TAC | CAC | | SEQ ID NO: 24 Y263H-R = |
| | | | | GCCAGGTGCAGCCCGTGAGCAAAAGTAGC |
| A354V | Alanine | Valine | 354 | SEQ ID NO: 25 A354V-F = |
| | GCA | GTA | | CTGGTTCGGCGATTACGTACAGCGTTTTCAGAGTG |
| | | | | SEQ ID NO: 26 A354V-R = |
| | | | | CACTCTGAAAACGCTGTACGTAATCGCCGAACCAG |
| T51A | Threo- | Alanine | 51 | SEQ ID NO: 27 ATOG-F = |
| | nine | GCA | | GGAGCGCGAGGCATCTATGTTGGCCTCGC |
| | ACA | | | SEQ ID NO: 28 ATOG-R = |
| | | | | GCGAGGCCAACATAGATGCCTCGCGCTCC |

TyrA mutagenesis was performed using the following recommended conditions:

### QuikChanqe® II Site-Directed Mutagenesis Kit Reaction Components:

5 μl 10 reaction buffer
2 μl plasmid DNA
125 ng forward primer
125 ng reverse primer
1 ul dNTP mix
38.5 μl h2O
1 ul PfuUltra DNA polymerase

### Thermalcycling Conditions:

95° C. 30 SEC

12 CYCLES OF

95° C. FOR 30 SEC

55° C. FOR 1 MIN

96° C. FOR 8 MIN
(2 min per 1 kb plasmid)

Each mutated coding region was transformed into XL1-Blue super-competent cells and plated overnight at 37° C. on LB agar plates containing 50 μg/mL kanamycin. Isolated kanamycin resistant colonies were then cultured at 37° C. overnight and the pENTR plasmid containing the mutated gene was isolated using QIAprep® Miniprep Kits (Qiagen Inc., Valencia, Calif., USA). All mutations were confirmed by automated DNA sequencing using the sequencing primers in Table 8 and ABI Prism BigDye™ Terminator Cycle Sequencing Version 3.1 Ready Reaction kit on the ABI 3700 Automated DNA Sequencer (Applied Biosystems, Foster City Calif.).

TABLE 8

Mutant coding region sequencing primers.

| | |
|---|---|
| Primer name | Sequence 5′ to 3′ |

| | | |
|---|---|---|
| TyrA2881F | CACTTTGTCCGTCACTG | SEQ ID NO: 29 |
| TyrA2897R | CAGTGACGGACAAAGTG | SEQ ID NO: 30 |
| TyrA3350F | GGCGTTTATTCAGGCAC | SEQ ID NO: 31 |
| TyrA3366R | GTGCCTGAATAAACGCC | SEQ ID NO: 32 |
| M13 Universal | GTAAAACGACGGCCAGT | SEQ ID NO: 33 |
| forward (−20) | | |
| M13 Universal | AACAGCTATGACCATG | SEQ ID NO: 34 |
| reverse (−24) | | |

The site directed mutations in tyrA were expressed from pDEW697, the vector with the E. coli IpdA strong promoter. Each site directed mutant tyrA coding region was shown to encode a functional prephenate dehydrogenase by the complementation of the tyrosine auxotrophy of E. coli AT2471. The expression plasmids with the mutant tyrA genes were tested in the E. coli K12-derived tyrosine producing strain, DPD4515. Shake flask experiments were conducted using cultures grown in a MOPS buffered defined medium with 2 g/L glucose and initial 10 μg/ml phenylalanine at 37° C. until glucose was depleted. The results of biological replicates are shown in Table 9.

TABLE 9
Tyrosine and phenylalanine produced by strains expressing TyrA
single mutants.

| Strain | TyrA | | Phe |
|---|---|---|---|
| name | Mutation(s) | Tyr (ppm) | (ppm) |
| DPD4604 | M53I/ | 311 | 38 |
| | A354V | | |
| DPD4604 | M53I/ | 311 | 36 |
| | A354V | | |
| DPD4605 | M53I | 277 | 47 |
| DPD4605 | M53I | 282 | 43 |
| DPD4606 | A354V | 311 | 35 |
| DPD4606 | A354V | 312 | 36 |
| DPD4607 | Y263H | 305 | 39 |
| DPD4607 | Y263H | 303 | 25 |
| DPD4608 | tyrA WT | 280 | 47 |
| DPD4608 | tyrA WT | 277 | 48 |

Tyrosine production was improved by 12% in the strains carrying the A354V mutation and by 9% in the strain carrying the Y263H mutation as compared with strains carrying the wild type tyrA in the same expression plasmid. The amount phenylalanine was also lower in the strains carrying these mutations. The M531 mutation alone did not result in improved tyrosine production.

Double and triple mutations were made in successive rounds producing the following mutants: M531/A354V, Y263H/A354V, Y263H/Q124R, and Y263H/A354V/Q124R, which included the combination of the two best tyrA mutants from the first set. These site directed mutations in tyrA were also expressed from pDEW697, the vector with the E. coli IpdA strong promoter. The expression plasmids with the mutant tyrA coding regions were tested in the E. coli K12-derived tyrosine producing strain, DPD4515. Shake flask experiments were conducted using cultures grown in a MOPS buffered defined medium with 2 g/L glucose and initial 10 μg/ml phenylalanine at 37° C. until glucose was depleted. The results are shown in Table 10.

TABLE 10
Tyrosine and phenylalanine produced by strains expressing
TyrA multiple mutants.

| | | Avg Tyr | Avg Phe |
|---|---|---|---|
| Strain name | gene on plasmid | (ppm) | (ppm) |
| DPD4515 | no plasmid | 333 | 76 |
| DPD4599 | tyrC | 398 | 75 |
| DPD4606 | tyrA(A354V) | 356 | 64 |
| DPD4608 | tyrA(WT) | 313 | 57 |
| DPD4622 | tyrA(Q124R) | 317 | 55 |
| DPD4623 | tyrA(Y263H-A354V-Q124R-) | 373 | 29 |
| DPD4624 | tyrA(Y2634-A534V) | 370 | 30 |
| DPD4625 | tyrA(Y263H-Q124R) | 348 | 47 |

The combination of two single mutations, which individually resulted in improved tyrosine production (A354V and Y263H), improved tyrosine production of DPD4515 by 19% as compared with wild type tyrA expressed from the same plasmid. Furthermore, this level of tyrosine production was moderately improved (4%) as compared with the best single mutation (A354V). This combination of mutations was not present in any of Lutke-Eversloh and Stephanopoulos mutants. However, in the same experiment, expression of the naturally feed back resistant Z. mobilis tyrC yielded substantially greater tyrosine production (8%) than the best tyrA double mutant.

### Example 6

### Additional Tyrosine Insensitive, Monofunctional Cyclohexadienyl Dehydrogenases

This example describes the identification of genes in other organisms that may encode tyrosine insensitive, monofunctional cyclohexadienyl dehydrogenases and thus may also be useful for improving tyrosine production and reducing phenylalanine.

It is known that other bacteria than Z. mobilis have cyclohexadienyl dehydrogenases (Bonner et al. (1990) Appl Environ Microbiol 56:3741-7). Some of these cyclohexadienyl dehydrogenases are inhibited by tyrosine and others, like that of Z. mobilis, are not inhibited by tyrosine (Xie et al. (2000) Comp Biochem Physiol & Toxicol Pharmacol 125:65-83). The Z. mobilis TyrC amino acid sequence was used to search for other putative tyrosine insensitive cyclohexadienyl dehydrogenases.

A BLASTP search of the GenBank non-redundant protein database using the TyrC amino acid sequence (SEQ ID NO:1) gave a score of 548 and an expectation value of 1×10⁻⁵⁴ to a protein annotated as a putative cyclohexadienyl dehydrogenase from Rhodopseudomonas palustris (ZP_{—}00847752). A protein annotated as a hypothetical protein from Agrobacterium tumefaciens strain C58 had a score of 526 and an expectation value of 1×10⁻⁵², and a protein annotated as prephenate dehydrogenase from Rhodospirillum rubrum had a score of 505 and an expectation value of 1×10⁻⁴⁹ in the same search. Other proteins are identified in searches such as this one, or using tBLASTN searching of DNA sequence databases.

To test whether proteins related to Z. mobilis TyrC are useful in increasing tyrosine production and decreasing phenylalanine production, the encoding sequences are cloned and expressed in a tyrosine production strain. For example, primers that are useful for amplification and directional cloning into the pENTR/SD/D-TOPO vector for three tyrc-related coding regions are:

Agrobacterium tumefaciens, strain C58

| | |
|---|---|
| Sense | CACCTGATGGGCGATATTATGTTT |
| (SEQ ID NO: 35) | |
| Antisense | TTATTTTTTCTGATCCAG |
| (SEQ ID NO: 36) | |

Rhodopseudomonas palustris

| | |
|---|---|
| Sense | CACCTGATGAACAGCGCGCCGATGT |
| (SEQ ID NO: 37) | |
| Antisense | TTATTCCGCGCCTTTGCC |
| (SEQ ID NO: 38) | |

Rhodospirillum rubrum

| | |
|---|---|
| Sense | CACCTGATGACCACCGCGCCGAGC |
| (SEQ ID NO: 39) | |
| Antisense | TTACGCCTGTTTCGCTTC |
| (SEQ ID NO: 40) | |

Following amplification, the PCR products are cloned into pENTR/SD/D-TOPO following the protocol from the manufacturer (Invitrogen). Subsequently, the LR Clonase reaction is used to move the cloned genes into an expression vector, such as pDEW697. Then tyrosine producing strains such DPD4009, DPD4515, or DPD4119 are transformed with the expression clones and tested for tyrosine and phenylalanine production in shake flasks in a MOPS-buffered minimal medium with 2 g/l glucose and initial 10 μg/ml phenylalanine. If the expressed coding region encodes an active cyclohexadienyl dehydrogenase, results show elevated tyrosine production and decreased phenylalanine production as compared with the parental strain without a plasmid. Furthermore, those with the most elevated tyrosine and/or decreased phenylalanine are candidates for a tyrosine insensitive, cyclohexadienyl dehydrogenase, which is confirmed by enzyme assay as described in General Methods.

1. An enhanced enteric tyrosine over-producing recombinant host cell comprising a genetic construct encoding a heterologus tyrosine insensitive prephenate dehydrogenase.

2. The enteric tyrosine over-producing recombinant host cell of claim 2 wherein the heterologus tyrosine insensitive prephenate dehydrogenase is a TyrC gene.

3. The enteric tyrosine over-producing recombinant host cell of claim 3 wherein the TyrC gene is isolated from the genera selected from the group consisting of Rhodopseudomonas, Rhodospirillum, and Agrobacterium and Zymomonas.

4-5. (canceled)

6. The recombinant host cell of claim 1 wherein the cell optionally comprises a non-functional pheA gene and an overexpressed tyrA gene.

7. The recombinant host cell of claim 6 wherein the cell optionally comprises a genetic trait selected from the group consisting of:
a) a feed back resistant DAHP synthase; and
b) a non-functional tyrR.

8. The recombinant host cell of claim 7 wherein the feed back resistant DAHP synthase comprises the aroG397 mutation.

9. The recombinant host cell of claim 7 wherein the non-functional tyrR has the tyrR366 mutation.

10. The recombinant host cell of claim 1 wherein the strain optionally comprises all of the following phenotypic traits:
a) resistance to 3-fluorotyrosine; and
b) resistance to para-fluorophenylalanine; and
c) resistance to β-2-thienylalanine; and
d) resistance to tyrosine; and
e) resistance to high phenylalanine and high temperature.

11. The recombinant host cell of claim 1 wherein the enteric bacteria is an E. coil.

12. The recombinant host cell of claim 11 wherein the E. coil is a strain selected from the group consisting of; TY1, DPD4009, DPD4515, DPD4119, and DPD4145.

13. A method for producing L-tyrosine comprising:
a) providing an enteric recombinant host cell according to claim 1 comprising a heterologus tyrosine insensitive prephenate dehydrogenase; and
b) growing said recombinant host cell under conditions where L-tyrosine is produced.

14. A method according to claim 13 wherein the enteric recombinant host cell comprises the following characteristics:
a) the presence of an aromatic amino acid biosynthetic pathway comprising genes selected from the group consisting of aroF, aroG, aroH, aroB, aroD, aroE, aroL, aroK, aroA, aroC, tyrA, pheA and tyrB
b) a non-functional pheA gene
c) overexpression of the tyrA gene;
d) resistance to 3-fluorotyrosine;
e) resistance to para-fluorophenylalanine;
f) resistance to β-2-thienylalanine;
g) resistance to tyrosine; and
h) resistance to high phenylalanine and high temperature.
